(19) United States
(12) Patent Application Publication
(10) Pub. No.: US 2024/0327830 A1
(43) Pub. Date: Oct. 3, 2024

(54) DISE-INDUCING SRNA-POLYPLEXES AND SRNA-LIPOPOLYPLEXES AND METHODS OF USING THE SAME TO TREAT CANCER

(71) Applicants: Northwestern University, Evanston, IL (US); University of Leipzig, Leipzig (DE)

(72) Inventors: Marcus Ernst Peter, Evanston, IL (US); Andrea E. Murmann, Evanston, IL (US); Achim Aigner, Leipzig (DE); Alexander Ewe, Leipzig (DE)

(21) Appl. No.: 18/176,126

(22) Filed: Feb. 28, 2023

Related U.S. Application Data
(60) Provisional application No. 63/268,694, filed on Feb. 28, 2022.

(51) Int. Cl. C12N 15/113; A61P 35/00

(52) U.S. Cl. CPC C12N 15/113; A61P 35/00; C12N 2310/321; C12N 2310/3515

(57) ABSTRACT

Disclosed are polynucleotides, compositions, and methods related to RNA interference (RNAi). In particular, disclosed are toxic RNAi sequences in polyplexes or lipopolyplexes and methods of using said the same for killing cancer cells and treating cancer.

### CROSS-REFERENCE TO RELATED APPLICATIONS

This application claims priority to U.S. Provisional Application No. 63/268,694, filed Feb. 28, 2022, which is incorporated by reference herein in its entirety.

### STATEMENT REGARDING FEDERALLY SPONSORED RESEARCH OR DEVELOPMENT

This invention was made with government support under CA197450 awarded by the National Institutes of Health. The government has certain rights in the invention.

### SEQUENCE LISTING

A Sequence Listing accompanies this application and is submitted as an xml file of the sequence listing named “702581_02292.xml” which is 8,901 bytes in size and was created on Feb. 27, 2023. The sequence listing is electronically submitted via Patent Center and is incorporated herein by reference in its entirety.

### BACKGROUND

Apart from surgery, most current, clinically used cancer therapies are based on four approaches: chemotherapy, radiation therapy, targeted therapy, and immunotherapy. These therapies all suffer from several shortcomings. These therapies may be toxic, prone to the acquisition of resistance, and not curative for all cancers. Successful treatments produce an objective response, but often extend life by only a few months. Therefore, there exists a need in the art for curative therapies with minimal side effects for the treatment of cancer.

### SUMMARY

In an aspect of the current disclosure, compositions are provided. In some embodiments, the compositions comprise: (1) a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19 A20 A21-3′
| | | | | | | | | | | | | | | | | | | | |
3′-B21 B20 B19 B18 B17 B16 B15 B14 B13 B12 B11 B10 B09 B08 B07 B06 B05 B04 B03 B02 B01-5′

wherein the region A02-A07 of the dsRNA is GGGGGC and, optionally, wherein the A and B strands comprise 3′ overhangs; (2) a polyamine-based polymer, wherein the dsRNA is in complex with the polyamine-based polymer and forms particles. In some embodiments, the A and/or B strand comprises modified nucleotides. In some embodiments, the nucleotides at positions B01 and B02 are modified. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In some embodiments, the compositions comprise: (1) a double-stranded polynucleotide comprising a passenger strand and a guide strand, the double-stranded polynucleotide comprising: a trinucleotide repeat sequence (X₁X₂X₃)ₙ, wherein the trinucleotide repeat of the guide strand is (CAG)ₙ, optionally, wherein the guide and passenger strand comprise 3′ overhangs, and (2) a polyamine-based polymer, wherein the dsRNA is in complex with the polyamine-based polymer and forms particles. In some embodiments, the passenger strand and/or guide strand comprises modified nucleotides. In some embodiments, the two most 5′ nucleotides of the passenger strand that are complementary to the guide strand are modified nucleotides. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In another aspect of the current disclosure, pharmaceutical compositions are provided. In some embodiments, the pharmaceutical compositions comprise: (1) a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19 A20 A21-3′
| | | | | | | | | | | | | | | | | | | | |
3′-B21 B20 B19 B18 B17 B16 B15 B14 B13 B12 B11 B10 B09 B08 B07 B06 B05 B04 B03 B02 B01-5′

wherein the region A02-A07 of the dsRNA is GGGGGC and, optionally, wherein the A and B strands comprise 3′ overhangs; (2) a polyamine-based polymer, wherein the dsRNA is in complex with the polyamine-based polymer and forms particles; and a pharmaceutically acceptable carrier. In some embodiments, the A and/or B strand comprises modified nucleotides. In some embodiments, the nucleotides at positions B01 and B02 are modified. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In some embodiments, the pharmaceutical compositions comprise: (1) a double-stranded polynucleotide comprising a passenger strand and a guide strand, the double-stranded polynucleotide comprising: a trinucleotide repeat sequence (X₁X₂X₃)ₙ, wherein the trinucleotide repeat of the guide strand is (CAG)ₙ, optionally, wherein the guide and passenger strand comprise 3′ overhangs, and (2) a polyamine-based polymer, wherein the dsRNA is in complex with the polyamine-based polymer and forms particles. In some embodiments, the passenger strand and/or guide strand comprises modified nucleotides. In some embodiments, the two most 5′ nucleotides of the passenger strand that are complementary to the guide strand are modified nucleotides. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In another aspect of the current disclosure, methods of treating a cell proliferative disease or disorder in a subject in need thereof are provided. In some embodiments, the methods comprise administering an effective amount of a pharmaceutical composition comprising (1) a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19 A20 A21-3′
| | | | | | | | | | | | | | | | | | | | |
3′-B21 B20 B19 B18 B17 B16 B15 B14 B13 B12 B11 B10 B09 B08 B07 B06 B05 B04 B03 B02 B01-5′

wherein the region A02-A07 of the dsRNA is GGGGGC and, optionally, wherein the A and B strands comprise 3′ overhangs; (2) a polyamine-based polymer, wherein the dsRNA is in complex with the polyamine-based polymer and forms particles; and a pharmaceutically acceptable carrier to treat the cell proliferative disease or disorder. In some embodiments, the A and/or B strand comprises modified nucleotides. In some embodiments, the nucleotides at positions B01 and B02 are modified. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation. In some embodiments, the cell proliferative disease or disorder is cancer. In some embodiments, the cancer is ovarian cancer. In some embodiments, the cancer is prostate cancer.

In some embodiments, the methods comprise administering a pharmaceutical compositions comprising: (1) a double-stranded polynucleotide comprising a passenger strand and a guide strand, the double-stranded polynucleotide comprising: a trinucleotide repeat sequence (X₁X₂X₃)ₙ, wherein the trinucleotide repeat of the guide strand is (CAG)ₙ, optionally, wherein the guide and passenger strand comprise 3′ overhangs, and (2) a polyamine-based polymer, wherein the dsRNA is in complex with the polyamine-based polymer and forms particles; and a pharmaceutically acceptable carrier. In some embodiments, the trinucleotide repeat of the guide strand is (CAG)ₙ. In some embodiments, the passenger strand and/or guide strand comprises modified nucleotides. In some embodiments, the two most 5′ nucleotides of the passenger strand that are complementary to the guide strand are modified nucleotides. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In another aspect of the current disclosure, methods of killing a cancer cell are provided. In some embodiments, the methods comprise contacting a composition comprising (1) a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19 A20 A21-3′
| | | | | | | | | | | | | | | | | | | | |
3′-B21 B20 B19 B18 B17 B16 B15 B14 B13 B12 B11 B10 B09 B08 B07 B06 B05 B04 B03 B02 B01-5′

wherein the region A02-A07 of the dsRNA is GGGGGC and, optionally, wherein the A and B strands comprise 3′ overhangs; (2) a polyamine-based polymer, wherein the dsRNA is in complex with the polyamine-based polymer and forms particles, to the cancer cell. In some embodiments, the A and/or B strand comprises modified nucleotides. In some embodiments, the nucleotides at positions B01 and B02 are modified. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation. In some embodiments, the cancer cell is an ovarian cancer cell, or derived from an ovarian cancer patient. In some embodiments, the cancer cell is a prostate cancer cell, or derived from a prostate cancer patient.

In some embodiments of the methods of killing cancer cells, the methods comprise contacting a composition comprising (1) a double-stranded polynucleotide comprising a passenger strand and a guide strand, the double-stranded polynucleotide comprising: a trinucleotide repeat sequence (X₁X₂X₃)ₙ, wherein the trinucleotide repeat of the guide strand is (CAG)ₙ, optionally, wherein the guide and passenger strand comprise 3′ overhangs, and (2) a polyamine-based polymer, wherein the dsRNA is in complex with the polyamine-based polymer and forms particles, to the cancer cell. In some embodiments, the trinucleotide repeat of the guide strand is (CAG)ₙ. In some embodiments, the passenger strand comprises modified nucleotides. In some embodiments, the modified nucleotides comprise 2′-O-methyl modifications. In some embodiments, the trinucleotide repeat of the guide strand is (CAG)ₙ. In some embodiments, the passenger strand and/or guide strand comprises modified nucleotides. In some embodiments, the two most 5′ nucleotides of the passenger strand that are complementary to the guide strand are modified nucleotides. In some embodiments, the modified nucleotides comprise 2′-O-methyl modifications. In some embodiments, the dsRNA and the polyamine-based polymer are present in polyplex particles. In some embodiments, the polyamine-based polymer comprises polyethyleneimine (PEI), optionally wherein the PEI is linear or branched, and wherein the average molecular weight of the polymer is 2,000-25,000 Da, preferably 5,000-20,000 Da. In some embodiments, the polyethyleneimine is modified with tyrosine. In some embodiments, 20-60%, 30-50%, or 30-35% of the primary amines in the polyethyleneimine are modified with tyrosine. In some embodiments, the composition further comprises (3) a lipid component, wherein the dsRNA and the polyamine-based polymer are present in lipopolyplexes (LPP). In some embodiments, the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC). In some embodiments, the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC). In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation. In some embodiments, the cancer cell is an ovarian cancer cell, or derived from an ovarian cancer cell. In some embodiments, the cancer cell is prostate cancer cell, or derived from a prostate cancer cell.

### BRIEF DESCRIPTION OF THE FIGURES

FIGS. 1A, 1B, 1C, and 1D: Activity of sRNAs delivered to both ovarian and prostate cancer cells in vitro. (A) HeyA8 cells expressing a Venus sensor (schematically shown above) that carries in its artificial 3′UTR 8 seed matches to the nontoxic seed present in the control sRNA sNT were left untreated or treated with either a control sRNA (sCtr) (sCAG used here as nontargeting control) or siNTI complexed in either PP or LPP particles. (C) the same experiment but using PC3 cells expressing eGFP and treated with siCtr (siLuciferase) or siGFP. Mean is shown −/+ variance. Student's t-test results are given. (B, D) Representative fluorescence images of the treated cells.

FIG. 2: Activity of toxic sRNAs delivered using PPs. HeyA8 cells expressing a Venus sensor carrying CUG TNRs in its 3′UTR (depicted above) were treated with the shown sRNAs for 48 hrs delivered as PP-sRNAs. Images were taken using a confocal microscope. Top row: Green fluorescence, bottom row: phase contrast. CUG CUG CUG CUG CUG CUG CUG CUG (SEQ ID NO: 1).

FIG. 3: Toxic sRNA-PPs kill multiple OC and PC cell lines. 2000-3000 human (green) or mouse (blue) OC or PC cells were incubated with 3-30 pmol of sRNA-PPs in 96 well plates and after 96 hrs viability was determined by ATP assay. N=sNT, 1=sGGGGGC, 2=sCAG. Shown is mean with SD and Student's t-test p-values. N=2-4; *** p<0.0001, ** p<0.001, * p<0.05. 60,577 cells are derived from Trp53^{-/-}-Brca1^{-/-}RB-TS inactive mice. TKO are organoids derived from Pten/Rb1/Trp53 triple knock-out (TKO) mice.

FIGS. 4A, 4B, 4C, 4D, 4E, and 4F: In vivo treatment with class 1 and class 2 sRNAs. PC3 prostate cancer cells were injected s.c. into nude mice and i.p. treated with sRNAs complexed in lipopolyplexes (LPP) (1). (A) sRNAs used; asterisks and red fonts indicate location of 2′-O-methylation modifications. sNT1 (UGGUUUACAUGUCGACUAdA (SEQ ID NO: 2) and UUAGUCGACAUGUAAACCAAA (SEQ ID NO: 3), sGGGGGC (UGGUUUACAUGUGCCCCCdA (SEQ ID NO: 4) and UGGGGGCACAUGUAAACCAAA (SEQ ID NO: 5), sCAG (GCUGCUGCUGCUGCUGCUdG (SEQ ID NO: 6) and CAGCAGCAGCAGCAGCAGCAG (SEQ ID NO: 7). (B) Treatment scheme. 21 days after s.c. implantation of 3×10⁶ PC3 cells (left and right flank) mice were treated i.p. with LPP-sNT1, LPP-siSurvivin, LPP-sGGGGGC, or LPP-sCAG (20 mg/mouse RNA). (C) Change in tumor volume over time. (D) Tumor weight at endpoint. Tumors from the same mouse (left and right) are linked by a vertical line. (E) H&E (left) and TUNEL (right) staining of four liver sections. (F) TUNEL quantification of livers (from indicated mice marked with an X) and quantification of liver enzymes (bottom) in treated mice. Numbers in brackets were likely outliers not related to treatment. neg, negative control; pos, positive control of tumor with cell death provided by core facility. * p<0.05, ** p<0.01.

FIGS. 5A, 5B, and 5C: Activity of sRNAs delivered to ovarian cancer cells in vitro. (A) Ranked list of genes in HeyA8 cells containing CUG TNRs of 19 nucleotides or longer. Genes in red were tested in B. (B) Normalized read numbers (CPM) of the top 5 most abundant genes in A in HeyA8 cells 48 hrs after transfected with either sNT1 or sCAG. (C) Change in confluence of HeyA8 cells transfected with either 10 nM of sNT1 or sCAG, or 20 nM of siSmartPools against the listed genes that carry CUG TNRs as shown in A. Knockdown of LRRC59 was not toxic (not shown).

FIGS. 6A, 6B, and 6C: Identification of most significantly downregulated target genes in HeyA8 and PC3 cells. HeyA8 or PC3 cells were transfected with either sNT1, sGGGGGC, or sCAG and 24/48 hours after transfection RNA was isolated and subjected to RNA Seq analysis. (A) GSEA of a set of genes containing GCCCCC target sites (the seed match for sGGGGGC) or CUG TNRs (the targeted sequence of sCAG) at least 10 nt long in their 3′UTR or mRNA, respectively. Genes were ranked according to highest downregulation induced by sGGGGGC or sCAG versus sNT1 at both time points. (B) 10 most highly expressed genes ranked according to average normalized reads in mock treated cells in either HeyA8 or PC3 cells carrying either GCCCCC seed matches or CUG repeats at least 10 nucleotides in length. Top 5 are shown in red. In case of the CUG TNR containing genes the length (in nucleotides) of the CUG repeat is given in brackets. (C) Normalized reads of the 5 most highly downregulated genes that carry the predicted target sequences (shown at the top) in cells transfected with the indicated sRNAs or just mock transfected. Only genes were included that had 1000 or more reads. The top five genes were then again ranked according to the abundance (also indicated by the grey triangle on the far right). Read numbers at the individual time points are shown. The sRNA that matches the analyzed target sequence is shown in red. Shared genes are shown in green.

FIGS. 7A, 7B, and 7C: Validation of target engagement by the two classes of sRNAs. PC3 cells were transfected with either 10 nM of siSurvivin (in vitro in A), sGGGGGC (in vitro in B), or sCAG (in vitro in C), each with sNT1 as nontargeting control, and after 48 hrs mRNA expression levels of Survivin, the most abundant target of sGGGGGC HMGA1 or the most abundant target of sCAG LRRC59, respectively, were quantified by real time PCR, relative to the expression of GAPDH. In addition, the right flank PC3 tumors of 7 mice treated with LPP-sNT1, LPP-siSurvivin, LPP-sGGGGGC, or LPP-sCAG were analyzed in the same way one day after the last treatment (see FIG. 4B). Student's t test p-values are shown.

FIGS. 8A, 8B, 8C, 8D, 8E, and 8F: Treatment of OC tumors orthotopically grown in syngeneic mice. (A) Treatment scheme. 10⁷ ID8 p53^{-/-}BRCA2^{-/-} cells were injected i.p. into female C57BL/6 mice and sRNA-LPP injections (red triangles) were also done i.p. (alternating injections on left and right side). (B) Tumor weight (histologically corrected) and mouse weight at endpoint. (C) TUNEL positivity in the liver and intestines of treated mice. (D, E) Analysis of the hematopoietic system (spleen and bone marrow [BM] isolated from tibia, femur, and iliac crest) in the treated mice. (F) Colony formation assay of BM cells. Three assays were set up for each of the three mice. LT-HSCs: Long-term hematopoietic stem cells; ST-HSCs: Short-term HSCs; MPPs: Multipotent progenitor cells; CMPs: Common myeloid progenitors; GMPs: Granulocyte and macrophage progenitors; MEPs: megakaryocyte and erythroid progenitors. Shown are the mean and ±SD (B and E) and the mean±SEM (D). ns, not significant in Ttest. sGGGGGC is abbreviated as sG5C.

FIGS. 9A, 9B, 9C, 9D, 9E, 9F, 9G, and 9H: Treatment of orthotopically implanted LC in immune competent male Sprague Dawley rats with LPP-sRNAs. (A) Viability (ATP content) of McA Rh7777 rat hepatoma cells in vitro treated with two nontoxic control sRNAs and the toxic sGGGGGC (sG5C) in LPPs for 96 hrs. (B) Treatment scheme of rats with LPP-sRNAs one week after surgical implantation of 2.5×10⁶ McA cells. (D) Tumor size in the liver was monitored by MRI. (C) Change in tumor volume over the treatment time. For one rat treated with sCtrl and one treated with sGGGGGC earlier MRI time points could not be analyzed due to technical issues with the MRI machine. (E, F) Representative H&E and TUNEL staining of liver sections of treated rats. (G) Quantification of TUNEL staining in the liver of rats treated with either of the ctr LPP-SRNAs (n=3) or LPP-sG5C (n=4). (H) Quantification of liver enzymes in the serum of treated rats.

### DETAILED DESCRIPTION

The present invention is described herein using several definitions, as set forth below and throughout the application.

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

Unless otherwise specified or indicated by context, the terms “a”, “an”, and “the” should be interpreted to mean “one or more.” For example, “an sRNA” should be interpreted to mean “one or more sRNA's”.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” should be interpreted to mean plus or minus ≤10% of the particular term and “substantially” and “significantly” should be interpreted to mean plus or minus >10% of the particular term.

As used herein, the terms “include” and “including” should be interpreted to have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, a “subject” may be interchangeable with “patient” or “individual” and means an animal, which may be a human or non-human animal, in need of treatment.

A “subject in need of treatment” may include a subject having a disease, disorder, or condition that can be treated by administering to the subject one or more therapeutic RNAs (e.g., a toxic RNA) formulated as RNA-polyplexes or RNA-lipopolyplexes as disclosed herein. A subject in need thereof may include a subject having or at risk for developing a cell proliferative disease or disorder such as cancer. A subject in need thereof may include, but is not limited to, a subject having or at risk for developing any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus). As such, methods of treating cancers are contemplated herein, including methods of treating cancers selected from, but not limited to any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus).

As used herein, a “toxic RNA” refers to an RNA molecule that induces cell death via RNA interference (RNAi) when the RNA molecule is introduced into or expressed in a cell. Toxic RNAs may include, but are not limited to, toxic sRNA, toxic pre-miRNA which may artificial or engineered pre-miRNA, and/or toxic miRNA (which may have been processed via Dicer from a corresponding pre-miRNA). Toxic RNAs have been disclosed in the art. (See U.S. Published Application Nos. 20180251762 and 20180320187, the contents of which are incorporated herein by reference in their entireties).

As used herein, the terms “silencing” and “inhibiting the expression of” refer to at least partial suppression of the expression of a target gene, for example, as manifested by a reduction of mRNA associated with the target gene.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art. As used herein, the term “therapeutically effective amount” refers to that amount of a therapeutic agent that provides a therapeutic benefit in the treatment, prevention, or management of a disease or disorder (e.g., a cell proliferation disease or disorder such as cancer).

As used herein, the term “pharmaceutical composition” may be defined as a composition that includes a pharmacologically effective amount of a toxic RNA and/or extracellular particles comprising the toxic RNA and a polyamine-based polymer, with or without addition of a lipid, e.g., liposome, component, wherein the dsRNA is in complex with the polyamine-based polymer and forms particles. A pharmaceutically composition as disclosed herein may include an additional acceptable carrier, diluent, or excipient.

### Polynucleotides

The disclosed technology relates to nucleic acid complexed with a polyamine-based polymer to form a nanoparticle. The nanoparticles (‘polyplexes’) may be further modified by adding a lipid component for generating a lipopolyplex. The disclosed technology further relates to the use of the nucleic acids in a polyplex or lipopolyplex for treating diseases and disorders. The terms “polyplex” and “complex”, are used interchangeably herein.

The terms “nucleic acid” and “oligonucleotide,” as used herein, refer to polyribonucleotides (containing ribose), polydeoxyribonucleotides (containing 2-deoxy-ribose), and to any other type of polynucleotide that is an N glycoside of a purine or pyrimidine base. As used herein, the terms “A,” “T,” “C,” “G” and “U” refer to adenine, thymine, cytosine, guanine, uracil as a nucleotide base, respectively. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded RNA. For use in the present invention, an oligonucleotide also can comprise nucleotide analogs in which the base, sugar or phosphate backbone is modified as well as non-purine or non-pyrimidine nucleotide analogs.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

As used herein, the term “complementary” in reference to a first polynucleotide sequence and a second polynucleotide sequence means that the first polynucleotide sequence will base-pair exactly with the second polynucleotide sequence throughout a stretch of nucleotides without mismatch. The term “cognate” may in reference to a first polynucleotide sequence and a second polynucleotide sequence means that the first polynucleotide sequence will base-pair with the second polynucleotide sequence throughout a stretch of nucleotides but may include one or more mismatches within the stretch of nucleotides. As used herein, the term “complementary” may refer to the ability of a first polynucleotide to hybridize with a second polynucleotide due to base-pair interactions between the nucleotide pairs of the first polynucleotide and the second polynucleotide (e.g., A:T, A:U, C:G, G:C, T:A, and U:A).

As used herein, the term “complementarity” may refer to a sequence region on an anti-sense strand that is substantially complementary to a target sequence or fully complementary to a target sequence. Where the anti-sense strand is not fully complementary to the target sequence, mismatches may be optionally present in the terminal regions of the anti-sense strand or elsewhere in the anti-sense strand. If mismatches are present, optionally the mismatches may be present in terminal region or regions of the anti-sense strand (e.g., within 6, 5, 4, 3, or 2 nucleotides of the 5′ and/or 3′ terminus of the anti-sense strand).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

As used herein, the term “double-stranded RNA” (“dsRNA”) refers to a complex of ribonucleic acid molecules having a duplex structure comprising two anti-parallel and substantially complementary nucleic acid strands.

As used herein, the term “nucleotide overhang” refers to an unpaired nucleotide or nucleotides that extend from the 5′-end or 3′-end of a duplex structure of a dsRNA when a 5′-end of one strand of the dsRNA extends beyond the 3′-end of the other strand, or when a 3′-end of one strand of the dsRNA extends beyond the 5′-end of the other strand. A nucleotide overhang may include ribonucleotides and/or deoxyribonucleotide (e.g., UU, dAdA or TT).

As used herein, the term “blunt” refers to a dsRNA in which there are no unpaired nucleotides at the 5′-end and/or the 3′-end of the dsRNA (i.e., no nucleotide overhang at the 5′-end or the 3′-end). A “blunt ended” dsRNA is a dsRNA that has no nucleotide overhang at the 5′-end or the 3′-end of the dsRNA molecule.

As used herein, the term “anti-sense strand” refers to a strand of a dsRNA which includes a region that is substantially or fully complementary to a target sequence (i.e., where the target sequence has a sequence corresponding to the sense strand).

As used herein, the term “sense strand,” refers to the strand of a dsRNA that includes a region that is substantially or fully complementary to a region of the anti-sense strand and that includes a region that substantially or fully corresponds to a region of the target sequence.

As used herein, RNAi active sequences may include “sRNA” and may also include dsRNA comprising a hairpin that is processed by nucleases to provide siRNA. sRNA may be loaded directly onto the RISC and do not require processing by DICER. The term “siRNA” refers to a “small interfering RNA”. RNA interference (RNAi) refers to the process of sequence-specific post-transcriptional gene silencing in a cell or an animal mediated by siRNA. As used herein, the term “sRNA” refers to short RNA, which may be double stranded (dsRNAs) and, as discussed above, do not require processing by DICER to be loaded onto the RISC.

As used herein, the term “siRNA targeted against mRNA” refers to siRNAs or sRNAs that specifically promote degradation of the targeted mRNA via sequence-specific complementary multiple base pairings (e.g., at least 6 contiguous base-pairs between the siRNA and the target mRNA at optionally at least 7, 8, 9, 10, 11, 12, 13, 14, 15, 16, 17, 18, 19, 20, 21, 22, or 23 contiguous base-pairings between the siRNA and the target mRNA).

As used herein, RNAi active sequences may include “pre-miRNA” and “miRNA” and dsRNA that is processed to provide pre-miRNA and miRNA. The term “pre-miRNA” refers to a “pre-micro RNA” and the term “miRNA” refers to “micro RNA.” RNA interference (RNAi) refers to the process of sequence-specific post-transcriptional gene silencing in a cell or an animal mediated by pre-miRNA and/or miRNA.

The terms “target, “target sequence”, “target region”, and “target nucleic acid,” as used herein, are synonymous and refer to a region or sequence of a nucleic acid which may be selected as a sequence to which the anti-sense strand of siRNA is substantially complementary to and hybridizes to as discussed herein. A target sequence may refer to a contiguous portion of a nucleotide sequence of an mRNA molecule of a particular gene, including but not limited to, genes that are essential for survival and/or growth of cells and in particular cancer cells. The target sequence of an siRNA refers to an mRNA sequence of a gene that is targeted by the siRNA due to complementarity between the anti-sense strand of the siRNA and the mRNA sequence and to which the anti-sense strand of the siRNA hybridizes when brought into contact with the mRNA sequence.

As used herein, the term “transfecting” means “introducing into a cell” a molecule, which may include a polynucleotide molecule such as dsRNA. When referring to a dsRNA, transfecting means facilitating uptake or absorption into the cell, as is understood by the skilled person. Absorption or uptake of dsRNA can occur or may be facilitated through passive diffusive or active cellular processes, and/or through the use of auxiliary agents or devices. Transfection into a cell includes methods known in the art such as electroporation, lipofection or other chemical methods triggering endocytosis, like using nanoparticles. However, the meaning of the term “transfection” is not limited to introducing molecules into cells in vitro. As contemplated herein, a dsRNA also may be “introduced into a cell,” where the cell is part of a living organism. For example, for in vivo delivery, a dsRNA may be injected into a tissue site or may be administered systemically.

### RNA Interference

The mechanism of action of RNA interference (RNAi) is understood by the skilled person. Interfering RNA (RNAi) generally refers to process that utilizes a single-stranded RNA (ssRNA) or double-stranded RNA (dsRNA) to inhibit expression of a target. The dsRNA is capable of targeting specific messenger RNA (mRNA) and silencing (i.e., inhibiting) the expression of a target gene. During this process, dsRNA (which may include pre-miRNA, or sRNA) is enzymatically processed into short-interfering RNA (siRNA) duplexes or miRNA duplexes by a nuclease called Dicer. The anti-sense strand of the siRNA duplex or miRNA duplex (referred to as the “guide strand”) is then incorporated into a cytoplasmic complex of proteins (RNA-induced silencing complex or RISC). The sense strand of the siRNA duplex of miRNA duplex (referred to as the “passenger strand”) is degraded. The RISC complex containing the anti-sense siRNA strand or anti-sense miRNA strand binds mRNA which has a sequence complementary to the anti-sense strand-allowing complementary base-pairing between the anti-sense strand and the sense mRNA molecule. The mRNA molecule is then specifically cleaved by an enzyme (RNase) associated with RISC called Argonaut 2 (Ago2) resulting in specific gene silencing. For gene silencing or knock down (i.e., mRNA cleavage) to occur, anti-sense RNA has to become incorporated into the RISC. This represents an efficient process that occurs in nucleated cells during regulation of gene expression.

In particular, siRNA-mediated RNA interference may be considered to involve two-steps: (i) an initiation step, and (ii) an effector step. The first step relies on input siRNA, which may have been delivered directly, e.g., sRNAs, or may have been generated intracellularly by the processing of longer dsRNA into small fragments by Dicer, e.g., dsRNAs comprising hairpins. These siRNAs comprise two strands that are ˜21-23-nucleotide in length. One strand is called “guide RNA.” The guide RNA, when incorporated into the protein-RNA RISC complex, is capable of degrading mRNA. As such, the RISC complex acts in the second effector step to destroy mRNAs that are recognized by the guide RNA through base-pairing interactions via Ago2. RNA interference may be considered to involve the introduction by any means of double stranded RNA into a cell which triggers events that cause the degradation of a target RNA, and as such may be considered to be a form of post-transcriptional gene silencing. The skilled person understands how to prepare and utilize RNA molecules in RNAi. (See, e.g., Hammond et al., Nature Rev Gen 2: 110-119 (2001); and Sharp, Genes Dev 15: 485-490 (2001), the contents of which are incorporate herein by reference in their entireties).

### Death Induced by Survival Gene Elimination

Previously, the inventors disclosed toxic RNAs that silence expression of one or more mRNAs of essential genes that are required for survival and growth of cells such as cancer cells. The disclosed toxic RNA molecules silence the expression of multiple mRNA's of essential genes that are required for survival and growth of cells such as cancer cells through a process similar to the process called “death-induced by survival gene elimination” or “DISE.” (See U.S. Publication Nos. 2018/0251762, 2018/0320187, 2020/0299694, and 2020/0299697, the contents of which are incorporated herein by reference in their entirety).

For purposes of this application, the anti-sense strand of the siRNA may comprise a contiguous nucleotide sequence, where the base sequence of the anti-sense strand has substantial or complete sequence complementarity to the base sequence of a contiguous nucleotide sequence of corresponding length contained in an mRNA sequence of the targeted mRNA (e.g., in a non-coding 3′-end of an mRNA sequence). Substantial complementarity permits some nucleotide mismatches (i.e., non-pairing nucleotides) and as such, the anti-sense strand of the siRNA need not have full complementarity.

At least a portion of an anti-sense strand of an siRNA molecule may comprise or consist of a sequence that is 100% complementary to a target sequence or a portion thereof. In another embodiment, at least a portion of an anti-sense strand of an siRNA molecule comprises or consists of a sequence that is at least about 90%, 95%, or 99% complementary to a target sequence or a portion thereof. For purposes of this application, the anti-sense strand of the siRNA molecule preferably comprises or consists of a sequence that specifically hybridizes to a target sequence or a portion thereof so as to inhibit expression of the target mRNA. The portion of the anti-sense strand of an siRNA molecule that comprises or consists of a sequence that is 100% complementary to a target sequence or a portion thereof may be a 6-nucleotide sequence referred to as a “seed sequence” which may be complementary to a corresponding 6-nucleotide sequence in a 3′ UTR of a mRNA of a survival gene. The complementarity in this 6-nucleotide seed sequence may be sufficient to induce “death-induced by survival gene elimination” or “DISE” as disclosed herein.

Methods for preparing and isolating siRNA or sRNA also are known in the art. The disclosed siRNA may be chemically synthesized, using any of a variety of techniques known in the art. The disclosed sRNA may include may contain one or more modified nucleotides, including one or more modified nucleotides at the 5′ and/or 3′ terminus of the RNA molecules. The disclosed RNA molecules may comprise one, two, three four or more modified nucleotides in the double-stranded region. Exemplary modified nucleotides may include but are not limited to, modified nucleotides such as 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, and the like. The preparation of modified sRNA is known by one skilled in the art. In some embodiments, the disclosed dsRNA molecules include one or more modified nucleotides at the 5′-terminus of the passenger strand of the dsRNA that prevent incorporation of the passenger strand into RISC. (See, e.g., Walton et al., Minireview: “Designing highly active siRNAs for therapeutic applications,” the FEBS Journal, 277 (2010) 4806-4813).

In one embodiment, the disclosed dsRNAs comprise a double stranded region of about 15 to about 30 nucleotides in length. Preferably, the disclosed RNAs are about 20-25 nucleotides in length. The disclosed RNAs of the present invention are capable of silencing the expression of a target sequence in vitro and in vivo.

In another embodiment, the dsRNA or sRNA has an overhang on its 3′ or 5′ ends. The overhang may be 2-10 nucleotides long. In one embodiment, the dsRNA or sRNA does not have an overhang (i.e., the dsRNA or sRNA has blunt ends).

In some embodiments, the disclosed sRNA molecules are capable of silencing one or more target mRNAs and may reduce expression of the one or more target mRNAs by at least about 20%, 30%, 40%, 50%, 60%, 70%, 80%, 90%, or 100% relative to a control sRNA molecule (e.g., a molecule not exhibiting substantial complementarity with the target mRNA). As such, in some embodiments, the presently disclosed sRNA molecules targeting the mRNA of essential genes may be used to down-regulate or inhibit the expression of essential genes (e.g., by at least about 20%, 30%, 40%, 50%, 60%, 70%, 80%, 90%, or 100% relative to a control sRNA molecule).

### dsRNA Polyplex and Lipopolyplex Compositions

The inventors discovered that combining dsRNAs used to trigger death induced by survival gene elimination (DISE) with a polyamine-based polymer, wherein the dsRNA is in complex with the polyamine-based polymer and forms particles (polyplexes), effectively killed prostate and ovarian cancer cells in vitro. Moreover, the disclosed polyplexes were effective at treating prostate and ovarian tumors in vivo in mouse models. Unexpectedly, lipopolyplexes based on the combination of these polyplexes with a liposome were found particularly efficient. Lipopolyplexes were found to better penetrate tissue and to more efficiently reach parts of the body that are hard to reach (so-called “deep compartments”). Lipopolyplexes may also benefit from being cellularly internalized by endocytosis processes different from their polyplex counterparts. Therefore, the disclosed polyplexes and lipopolyplexes represent a novel cancer therapeutic for the treatment of cancer, e.g., prostate and ovarian cancers.

As used herein, the term “polyplex” refers to a nanoparticle, e.g., in the range of 50-1000 μm comprising a polyamine-based polymer, e.g., a polyethylenimine, with or without chemical modifications, and a polynucleotide as defined above. There is no intended distinction between “polyplex” and “complex”, and these terms will be used interchangeably.

As used herein, the term “lipopolyplex” refers to the combination of a polyplex with a lipid component, for example a liposome, leading to a nanoparticle comprising all three of the said components.

As used herein, the term “complexation” refers to the process of combining the complex components in a way that defined nanoparticles are formed. In addition, complexation refers to the act of generating lipopolyplexes from, e.g., polynucleotides, polyamine-based polymers, and a lipid component, e.g., a liposome.

Accordingly, in one aspect of the current disclosure, compositions are provided. In some embodiments, the compositions comprise: (1) a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19 A20 A21-3′
| | | | | | | | | | | | | | | | | | | | |
3′-B21 B20 B19 B18 B17 B16 B15 B14 B13 B12 B11 B10 B09 B08 B07 B06 B05 B04 B03 B02 B01-5′,

the dsRNA defined as follows: A01 through A21 and B01 through B21 are any ribonucleotide selected from A, U, G, and C, provided that: (i) A01-A21 are complementary to B01-B21; (ii) A01 and B01 are A or U; (iii) the percentage GC content of the region from A02-A07 (B15-B20) is 66-100% (Class 1 DISE-inducing sRNA); and (2) a polyamine-based polymer, wherein the dsRNA is in complex with the polyamine-based polymer and forms particles.

In some embodiments, the region A02-A07 of the dsRNA is GGGGGC. In some embodiments, the A and/or B strand comprises modified nucleotides. In some embodiments, the modified nucleotides comprise 2′-O-methyl modifications. In some embodiments, the region A02-A07 of the dsRNA is GGGGGC and B01 and B02 comprise modified nucleotides, e.g., 2′-O-methylated nucleotides.

In some embodiments, the compositions comprise: (1) a double-stranded polynucleotide comprising a passenger strand and a guide strand, the double-stranded polynucleotide comprising: a trinucleotide repeat sequence (X₁X₂X₃)ₙ, wherein X₁, X₂, and X₃ independently are selected from any ribonucleotide A, C, G, and U, and n is an integer from 3-10 (Class 2 DISE-inducing sRNA); and (2) a polyamine-based polymer, wherein the dsRNA is in complex with the polyamine-based polymer and forms particles. In some embodiments, the trinucleotide repeat of the guide strand is (CAG)ₙ. In some embodiments, the A and/or B strand comprises modified nucleotides. In some embodiments, the modified nucleotides comprise 2′-O-methyl modifications. In some embodiments, the trinucleotide repeat of the guide strand is (CAG)ₙ and the passenger strand comprises modified nucleotides, e.g., 2′-O-methylation.

In some embodiments, the dsRNA and the polyamine-based polymer are present in polyplex particles.

As used herein, “polyamine-based polymer” refers to a polymer comprising individual monomers which comprise two or more nitrogen containing groups or moieties. In some embodiments, exemplary polyamine-based polymers are polyethyleneimine (PEI). In some embodiments, exemplary polyamine-based polymers are low molecular weight polyamine-based polymers. In some embodiments, the polyamine-based polymer has an average molecular weight of 2,000-25,000 Da, preferably 5,000-20,000 Da. In some embodiments, the polyamine-based polymer comprises polyethyleneimine (PEI), wherein PEI is linear or branched. In some embodiments, the polyamine-based polymer is modified with an amino acid, e.g., tyrosine via conjugation. As used herein, “modified with tyrosine” refers to the modification of primary and/or secondary amines on the polyamine-based polymer to comprise tyrosine. See, for example, Creusat and Zuber, “Tyrosine-modified PEI: a novel and highly efficient vector for siRNA delivery in mammalian cells”. Nucleic Acids Symp Ser (Oxf). 2008; (52):91-2. The degree of tyrosine modification, also described as “grafting” or the “degree of grafting” is described in terms of a percentage which, as used herein, describes the percentage of primary amines on PEI that is modified with tyrosine. The degree of tyrosine grafting may be 20-60%, preferably 30-50%, or most preferably 30-35%.

In some embodiments, the composition further comprises (3) a lipid component, wherein the dsRNA and the polyamine-based polymer are present in lipopolyplexes (LPP). The lipid component of the disclosed lipopolyplexes may be, for example, 1,2-dipalmitoyl-sn-glycero-3-phosphocholine (DPPC).

The lipid components described above may be in the form of a liposome, for example. Thus, the disclosed lipopolyplexes may be generated by the complexation of (1) polynucleotides, (2) polyamine-based polymer, and (3) a lipid component, wherein the lipid component is a liposome.

In some embodiments, the composition is prepared in HEPES buffered glucose, HEPES buffered trehalose, HEPES buffered NaCl. In some embodiments, the composition is prepared in glucose or trehalose with a concentration in the range from 0.1 mM to 10 mM. In some embodiments, the z-average size of the particles is less than 800 nm, preferably less than 500 nm. In some embodiments, the particles are neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. In some embodiments, the z-average size of the particles and/or the Zeta-potential are determined by the conditions of polyplex or lipopolyplex formation.

In some embodiments, the zeta potential of the lipopolyplexes has a range of about-5 mV to about 10 mV. However, it will be understood by one of skill in the art that the specific zeta potential of the lipopolyplexes of the instant disclosure depend on the parent polyplex, the amount of lipid used to generate the lipopolyplexes, and the buffer used.

In addition to the above components, the compositions of the current disclosure may also, include further components which stabilize and/or add to the biological activity of the disclosed compositions. For example, the process needed to generate the nanoparticles may use HEPES buffered glucose, HEPES buffered trehalose, HEPES buffered NaCl. The complexation process may involve the use of glucose or trehalose with a concentration in the range from 0.1 mM to 10 mM. The z-average size of the particles may be less than 800 nm, preferably less than 500 nm. The particles may be neutral or positively charged, preferably at physiological pH or at a pH between 4.5 and 7.5. In some embodiments, the Zeta-potential of the particles is 0-30 mV or more, preferably 10 mV-25 mV in the case of polyplexes and −5 mV-10 mV in the case of lipopolyplexes. The z-average size of the particles and/or the Zeta-potential are, suitably, determined by the conditions of polyplex or lipopolyplex formation.

### Pharmaceutical Compositions

In another aspect of the current disclosure, pharmaceutical compositions are provided. In some embodiments, the pharmaceutical compositions comprise: the polyplexes or lipopolyplexes of the current disclosure; and a pharmaceutically acceptable excipient or diluent.

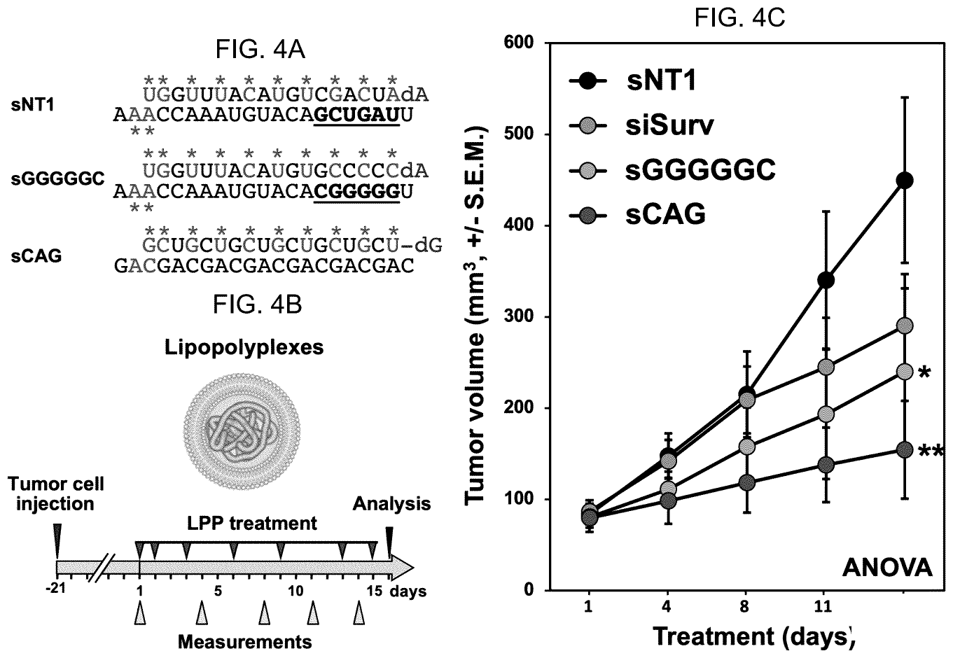

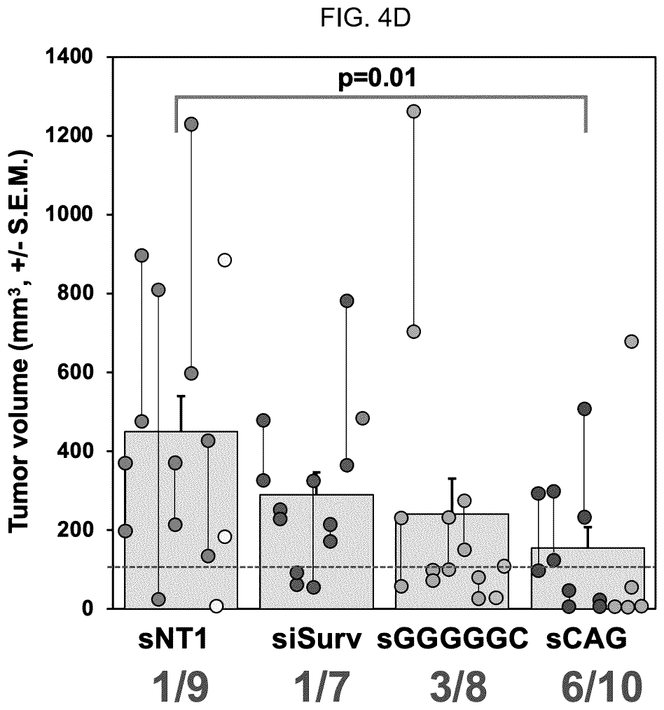

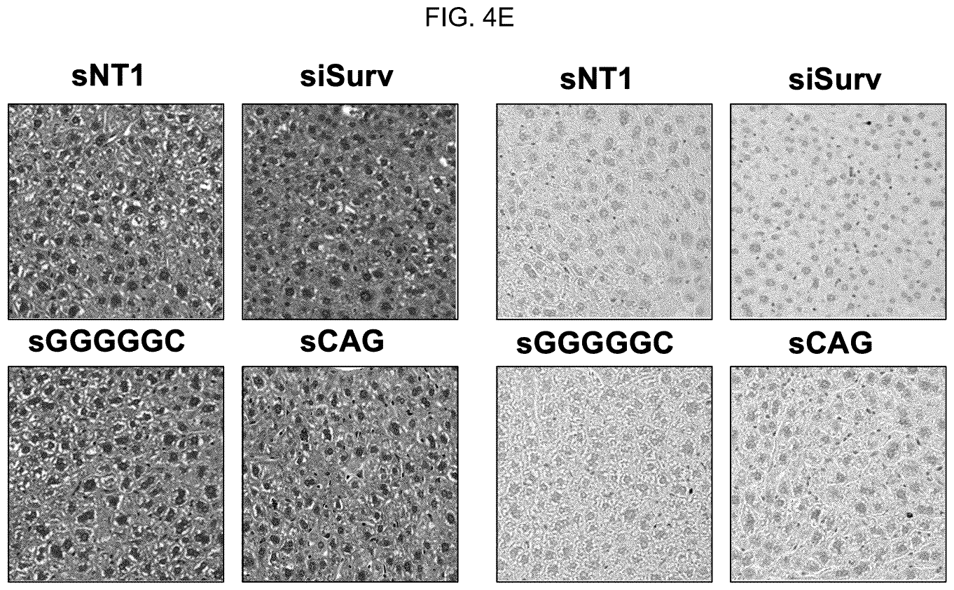

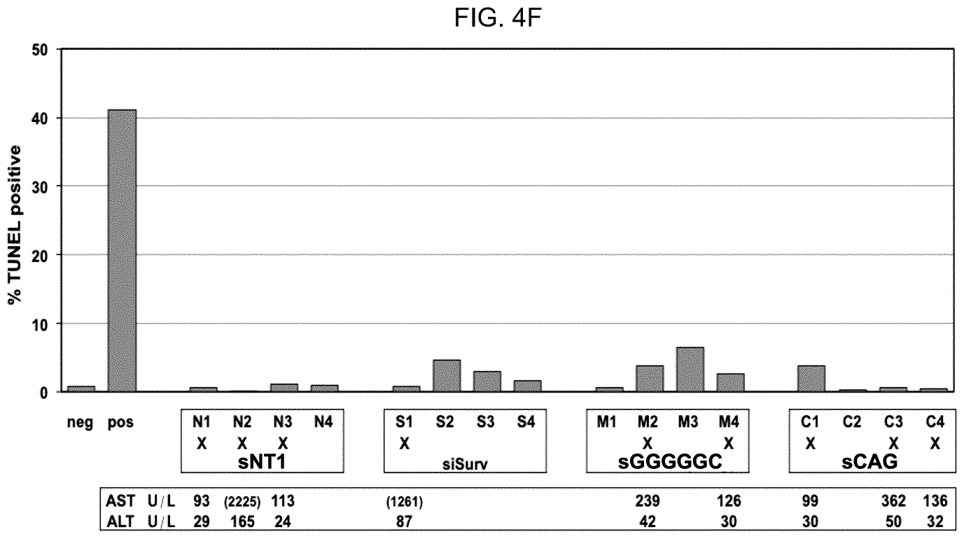

The pharmaceutical compositions may be formulated for administration intravenously, intranasally, intramuscularly, subcutaneously, intraperitoneally, rectally, intratumorally, or any other suitable route. For example, the inventors have demonstrated that formulation of the disclosed pharmaceutical compositions for intraperitoneal administration is effective in the treatment of a prostate cancer using a mouse model (FIG. 4).

### Methods of Treatment

The inventors discovered that administration of the disclosed sRNA via polyamine-based polymer complexes to animals with prostate or ovarian tumors significantly reduced tumor growth, or in some cases, cured the mice of the tumors. Furthermore, the inventors discovered that the disclosed methods are effective in treating cell proliferative diseases or disorders by selectively killing cancer cells, while sparing normal or non-malignant cells.

Therefore, in another aspect of the current disclosure, methods of treating a cell proliferative disease or disorder in a subject in need thereof are provided. In some embodiments, the methods comprise administering an effective amount of the disclosed pharmaceutical compositions to treat the cell proliferative disease or disorder.

In another aspect of the current disclosure, methods of killing a cancer cell are provided. In some embodiments, the methods comprise contacting a composition comprising the disclosed polyplexes and/or lipopolyplexes to the cancer cell.

In some embodiments, the cancer cell is an ovarian cancer cell, or derived from an ovarian cancer patient. In some embodiments, the cancer cell is a prostate cancer cell, or derived from a prostate cancer patient.

### Examples

The following Examples are illustrative and are not intended to limit the scope of the claimed subject matter.

### 1. Overview

We identified a new RNAi based form of cell death induction that is effective against all cancers (2-10). Cancer cells cannot become resistant, and it does not affect normal tissues (6, 9). We found that certain short (s)RNAs when loaded into the RNA induced silencing complex (RISC) are toxic to cancer cells by targeting a network of genes that are critical for cell survival, a process we have named DISE (for death induced by survival gene elimination) (7, 8). DISE involves simultaneous activation of multiple cell death mechanisms (11). Cancer cells cannot become resistant to DISE as it involves targeting a large network of genes, what likely covers all cells even in a heterogeneous tumor. On the other hand, we have not seen any signs of toxicity in non-tumor cells and thus also no signs of toxicity of the treatment in mice.

We have discovered two different mechanisms of DISE like activities that resulted in the development of two classes of sRNAs that can be used for therapy: Class 1 sRNAs carry a G-rich 6mer seed region that targets C-rich seed matches mainly located in the 3′ UTR of genes in a miRNA-like manner (7, 10). This was discovered by performing an arrayed screen of all 4096 possible 6mer seeds placed in a neutral sRNA backbone after inactivation of the passenger strand on three human and three mouse cell lines (2, 7). The most toxic seeds were high in Gs. Such toxic G-rich sequences were then found in a number of highly conserved tumor suppressive miRNAs ((7, 12) and 6merdb.org)). They kill through 6mer Seed Toxicity we discovered, a mechanism that induces DISE (7).—U.S. Ser. No. 15/900,392 & PCT/US2018/018801, filing Date: Feb. 20, 2018.

Class 2 sRNAs are derived from trinucleotide repeat (TNR) regions that when extended can cause disease (5, 6). The most potent TNR, CAGₙ, is found in Huntington's disease (HD) patients, who, interestingly, have a profoundly reduced cancer incidence. CAG-based sRNAs kill cells by targeting a network of genes that carry CUG TNRs in an sRNA-like manner and our analysis again confirmed that many of the targeted genes are survival genes (6). Class 2 sRNAs therefore kill cells through a DISE-like mechanism but they are up to 100 times more potent in killing cancer cells than class 1 sRNAs.—US patent issued U.S. Pat. No. 10,934,547 (Mar. 2, 2021) PCT/US2018/018798.

Ovarian carcinoma (OC) is one of the deadliest cancers affecting women and most patients develop resistance to any type of therapy, most notably platinum (Pt) based therapy which is part of the first line treatment of ovarian cancer. Prostate carcinoma (PCa) is the most common cancer specific to men and advanced castration resistant prostate cancer does not respond to any therapy anymore. We have developed a radically new form of therapy for these deadly cancers that are specific to either women or men by combining the two most toxic sRNAs, sGGGGGC (also referred to as sG5C) (class 1) and sCAG (class 2) with polyplexes (PP) and lipopolyplexes (LPP) to effectively deliver the toxic sRNAs to cancer cells. We show that we can deliver RNAi active sRNAs using PPs and LPPs to both ovarian and prostate cancer cells in vitro and we have treated prostate cancer in a preclinical mouse model using LPP-sGGGGGC and LPP-sCAG in vivo. We have identified the targets of the two sRNAs in both ovarian cancer and prostate cancer cells and provide evidence of target engagement in treat tumors. Finally, we provide evidence that neither of the treatments caused any toxicity in the treated mice suggesting that our toxic class 1 and class 2 sRNAs complexed as PPs or LPPs can be used to treat cancer.

### 2. Treatment of Ovarian Cancer and Prostate Cancer with Class 1 and Class 2 LPP-sRNAs

### 2.1 In Vitro Testing

The polyethyleneimine (PEI)-complexation of nucleic acids provides a non-viral delivery platform with in vivo efficacy. In the Aigner group, PEI-based nanoparticles (‘complexes’, ‘polyplexes’ (PP)) have been developed for the delivery of small RNAs. This included the development of more efficient and less toxic low molecular weight PEIs and novel chemical modifications thereof.

PEIs are positively charged polymers that form non-covalent complexes with nucleic acids, thus (i) protecting small RNAs from degradation, (ii) mediating cellular uptake, and (iii) efficiently promoting lysosomal protection and escape into the cytoplasm (13). The Aigner group has previously established the 4-12 kDa branched PEI F25-LMW, a low-molecular weight PEI with superior transfection efficacy and low toxicity, for delivery of small RNA molecules in vivo and in vitro (14-17). Thereby, we successfully altered miRNA levels in vivo by complexing synthetic miRNA mimics (small modified double-stranded miRNA molecules) with PEI (17, 18).

For further improvement of efficacy and biocompatibility, studies were extended towards the combination of polymeric, PEI-based polyplexes with liposomes, leading to lipopolyplexes (LPP) that combine the advantages of both systems. This further modification of established polyplexes with liposomes has been demonstrated as very efficient in vivo, including in the brain (19, 20).

Alternatively, the introduction of tyrosine-modifications to the PEI polymers led to increased complex stability especially in the case of very small branched or linear PEIs, and markedly improved transfection/knockdown efficacy as well as improved biocompatibility (21-24). Notably, enhanced efficacy includes, in particular, hard-to-transfect cells and favorable tissue penetration. Again, these systems have also been investigated in subcutaneous and orthotopic xenograft models in vivo studies, for therapeutic intervention (23, 24). High efficacy, in combination with favorable biocompatibility, was observed.

Thus, PPs and LPPs can be used to deliver small RNAs to tumor cells in vivo (16, 17, 25). This also includes the setting of prostate cancer. For example, we recently published that delivery of miR-143 using polyplexes (PP) and lipopolyplexes (LPP) caused substantial slowdown in xenografted PC3 prostate cancer cells in vivo (1).

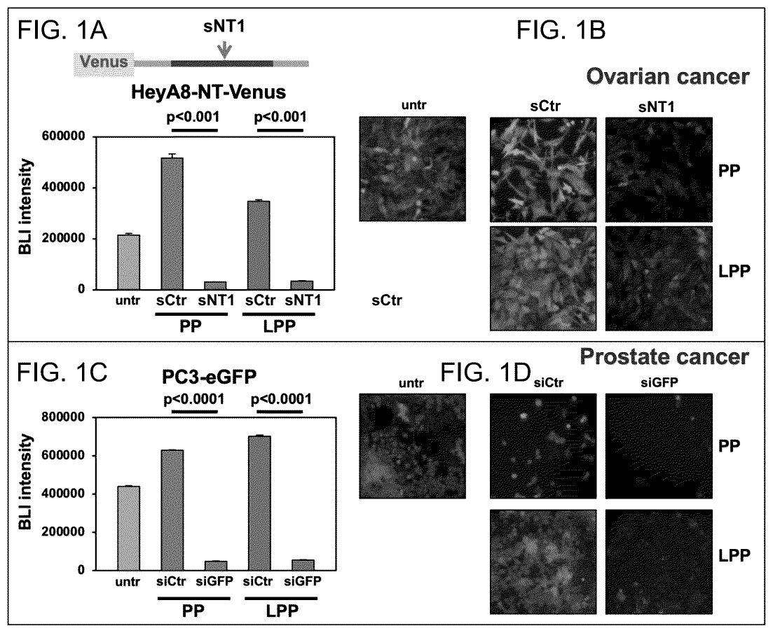

These particles are also very effective to deliver the DISE-inducing sRNAs to cancer cells in vitro and in vivo (FIGS. 1-4). For in vitro studies, we first chose two different cancer cell lines. First, we treated the ovarian cancer cell line HeyA8 expressing a Venus fluorescence sensor that allows to quantify the level of silencing by the control sRNA sNT1 that targets motifs in the 3′UTR of that Venus sensor. sCAG was used as nontargeting control in this experiment. Both PPs and LPPs were used. The data demonstrate that both LPP-sNT and LPP-sNT was highly effective in silencing Venus fluorescence (FIG. 1A). This confirmed that the particles were taken up by the cells and reached their targets. In fact, almost all cells took up the particles equally as documented by the reduction in fluorescence in the treated cells (FIG. 1B). Because we are interested in both ovarian and prostate cancer, a similar experiment was performed using the AR negative cell line PC3 (FIGS. 1C, 1D). In this case GFP expressing cells were used and treated with siLuciferase as nontargeting control and siGFP again complexed in either PPs or LPPs. The results were virtually identical to the ones obtained with the ovarian cancer cell line suggesting that the sRNA delivery can be used to deliver sRNA to a variety of cancers.

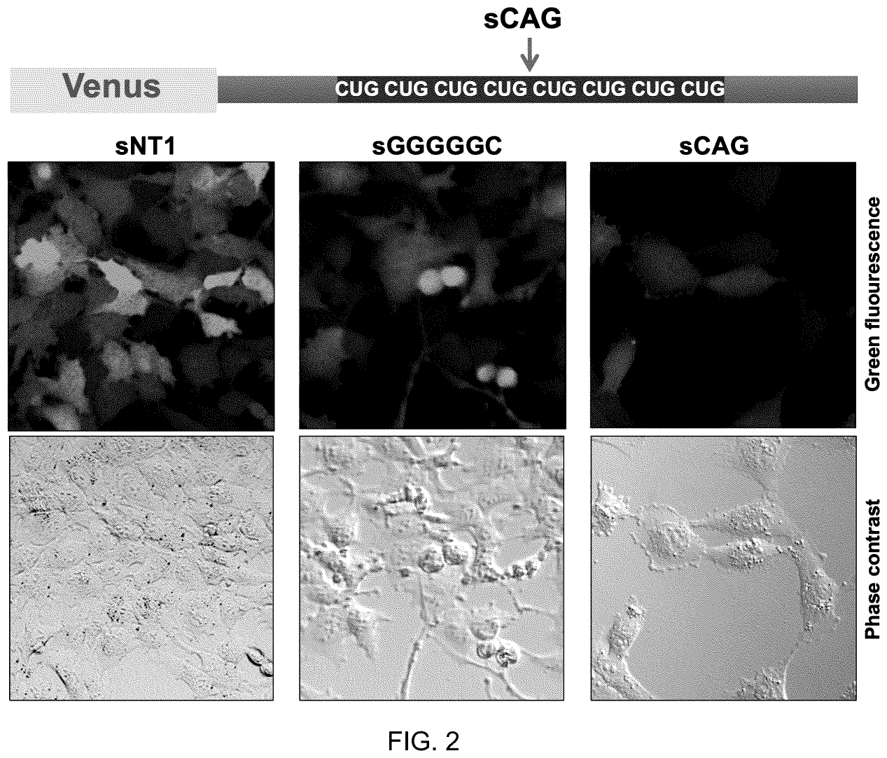

To determine whether a toxic sRNA could be delivered to cancer cells, engaged a target and causes signs of cell death, we treated HeyA8 cells expressing a Venus sensors designed to respond to sCAG with either sNT1, the class 1 sRNA sGGGGGC, or sCAG (FIG. 2). SCAG suppressed Venus fluorescence and after this relatively short incubation time of 48 hrs cells showed first signs of cell death with small blebs appearing on the cell surface. In cells treated with sGGGGGC cells started to crumble up and die without losing green fluorescence as the sensor was not designed to respond to this sRNA. In summary, the data suggest that both toxic sRNAs can be delivered to cancer cells using (L)PPs slowing down cell growth and inducing signs of cell death.

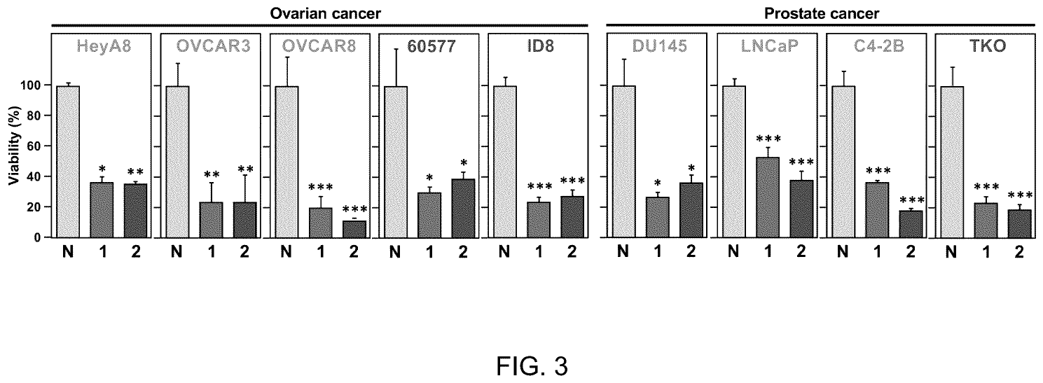

Finally, we delivered either class 1 or class 2 sRNAs using PPs to various ovarian and prostate cancer cell lines and quantified cell death after 96 hrs of exposure using an ATP assay (FIG. 3). All tested cell lines, both of human and mouse origin showed reduction in viability upon treatment with either of the two toxic sRNA-PPs.

### 2.2. In Vivo Treatment

Prostate cancer was selected as the first model in which to test the effectiveness of the two toxic sRNAs complexed in LPPs. One reason was that prostate cancer is a cancer in which DISE/6mer seed toxicity concept was completely confirmed and described as androgen network DISE (AN-DISE) (26). This study showed that in prostate cancer targeted survival genes are dominated by AR regulated genes. In this experiment we compared for the potency of class 1 and class 2 sRNAs with one another and with the targeting of a single established oncogene in the same mouse tumor model. The toxic sRNAs and sNTI were chemically modified in a way that increases stability in vivo, reduces innate immune activation and completely blocks uptake of the passenger strand into the RISC (FIG. 4A). Mice were s.c. injected with PC3 cells and after establishment of tumors (21 days) they were i.p. treated with LPP-sNT1, LPP-siSurvivin, LPP-sGGGGGC (class 1), and LPP-sCAG (class 2) (FIGS. 4A, 4B). siSurvivin had some effect on tumor growth, sGGGGGC was more effective and sCAG was most potent (FIGS. 4C, 4D). In fact, 6/10 mice treated with LPP-sCAG showed no tumor growth and four of these were completely cured of tumors (FIG. 4D). Most remarkable was the fact that the treated mice showed no signs of toxicity. Weight of the mice was not affected (not shown), we did not see any pathological changes in liver morphology (FIG. 4E, left) or a significant increase in liver enzymes (FIG. 4F, bottom). In addition, no significant increase in TUNEL positivity of livers, as a measure of cell death in the treated mice, was detected (FIG. 4E, right and FIG. 4F, top).

Together with our published studies on the delivery of class 1 and class 2 siRNAs to ovarian cancer (6, 9) and our studies on the treatment of ovarian cancer with class 1 shRNAs, these new data strengthen our conclusion that the toxic s/shRNAs act independent of the type of cancer or delivery method and are not toxic to treated mice.

### 2.3. Identifying the Targets in Class 1 and Class 2 sRNA Treated Ovarian Cancer and Prostate Cancer

Next, we set out to confirm target engagement in the treated mice. We identified the targeted genes in the two cancer cell lines HeyA8 and PC3. Because our treatment is not targeting a single gene but a different set of genes in every cancer, in fact most likely different in every single cancer cell, we can identify the most likely targets by determining which of the genes that carry either GCCCCC seed matches in their 3′UTR or CUG TNRs in the mRNA are highly expressed in a given tumor or tissue.

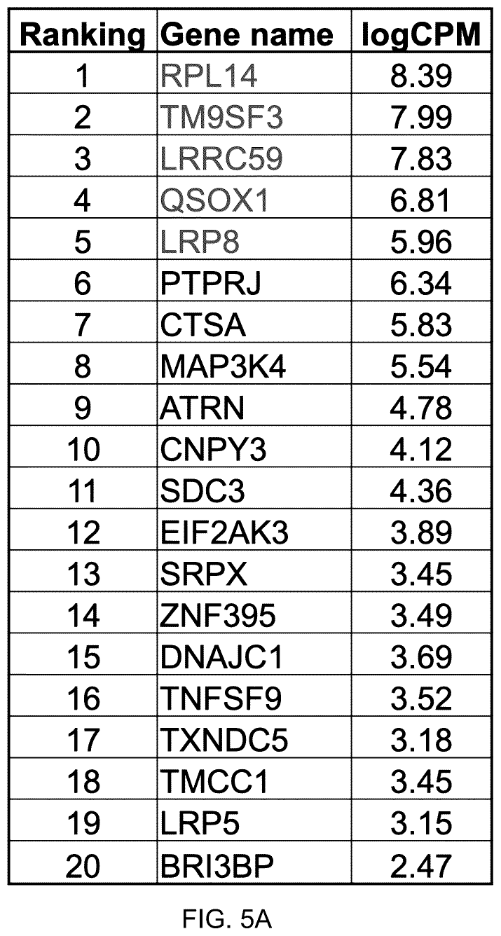

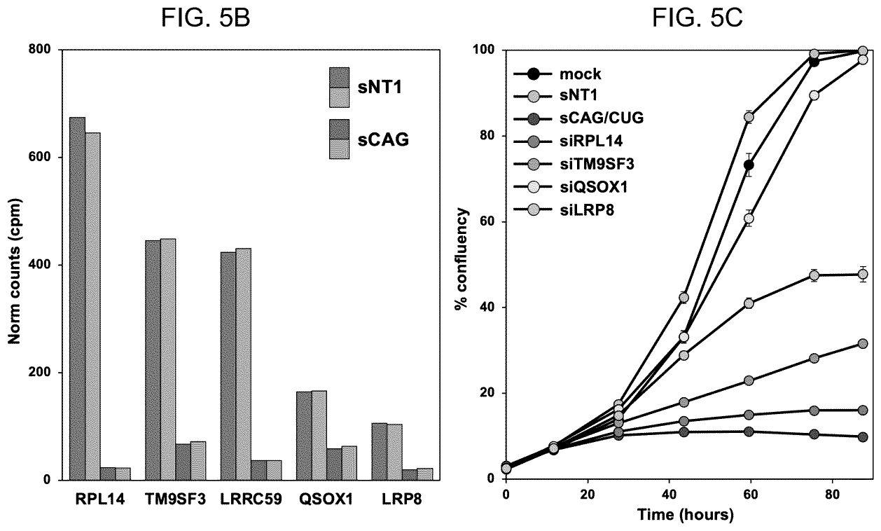

Another goal was to test whether silencing the most abundant genes that are most highly knocked down by a toxic siRNA using an siSmartPool that does not target the CUG TNR will also result in death of the cells. This would provide evidence for the toxic sRNA killing through silencing this network of genes. This was done for HeyA8 cells treated with sCAG. HeyA8 cells were transfected with sNT1 or sCAG and after 48 hrs RNA was isolated and subjected to an RNA sequencing (RNA-seq) analysis. The genes that carried CUG TNRs in their mRNA at least 19 nts in length were ranked according to highest expression in control treated cells (FIG. 5A). The normalized reads of the top five most highly expressed genes was plotted from cells treated with either sNT1 or sCAG (FIG. 5B). All five genes were fundamentally downregulated by sCAG. To test whether the silencing of these genes could explain why the cells died, HeyA8 cells were transfected with 20 nM of siSmartPools against these five genes and analyzed in the IncuCyte (FIG. 5C). With the exception of LRRC59 (not shown) knock down of the other four genes resulted in reduced cell growth with the knockdown of the most abundant genes being most toxic. Treating the cells with an siRNA SmartPool against the most highly expressed gene RLP14 was almost as toxic as treating the cells with sCAG (FIG. 5C). Interestingly, RPL14 which is also highly abundant in prostate cancer cells is part of a set of genes we had previously defined a critical survival genes for all cells (8).

To obtain a list of genes that are significantly expressed, contain predicted target sites and are selectively downregulated by the toxic sRNAs in either ovarian cancer or prostate cancer cells in a sustained way, we transfected both HeyA8 and PC3 cells with the nontargeting control siRNA sNT1, sGGGGGC, or sCAG, all chemically modified in the way shown in FIG. 4A. Cells treated with transfection reagent only (mock) were also added as controls. RNA was isolated from the cells either 24 or 48 hours after transfection. The rationale to use different time points rather than duplicates was that only genes should be considered that maintained a robust downregulation over at least one day. We generated lists of all human genes that contain at least one GCCCCC sequence in their 3′UTR (the longest transcript was used for each gene) and all genes that contained a CUG repeat in their mRNA that was at least 10 nt in length. We extended the length of the sCAG targeted sequence as it should be possible for sCAG to target CUG repeats shorter than 19 nts. Again, the longest composite transcript was used for each gene.

Using the mock treated samples, for each cell line we identified a list of genes that contained the predicted target sites and that were expressed at a 1000 reads or more in the mean of the two mock treated samples (using data sets normalized to one million reads). 1929 GCCCCC containing targets that met these criteria were identified for HeyA8 cells and 2030 for PC3. 421 CUG TNR targets were identified in HeyA8 cells and 455 in PC3 cells.

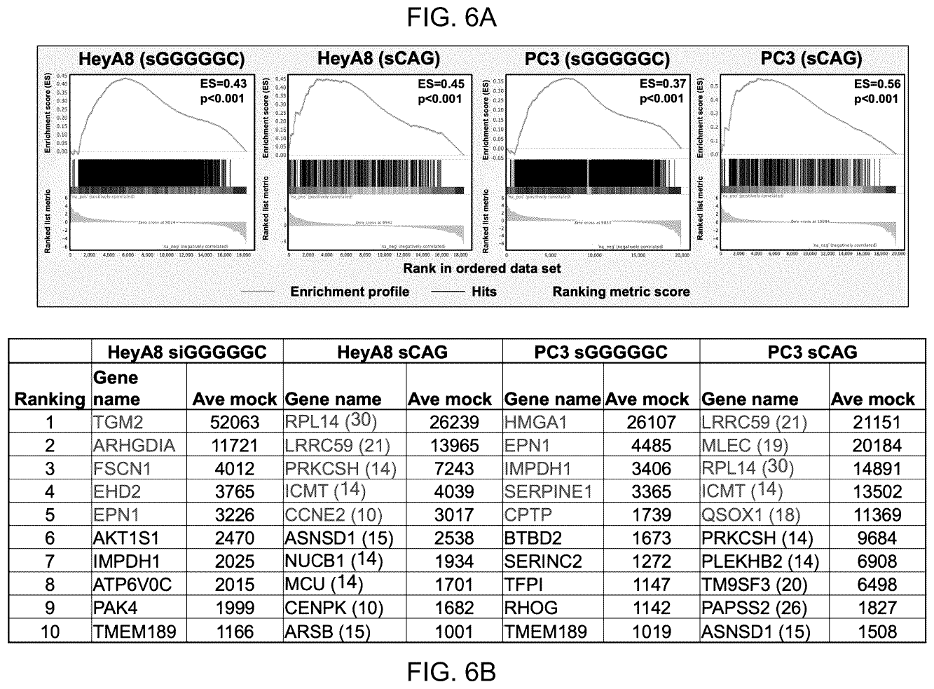

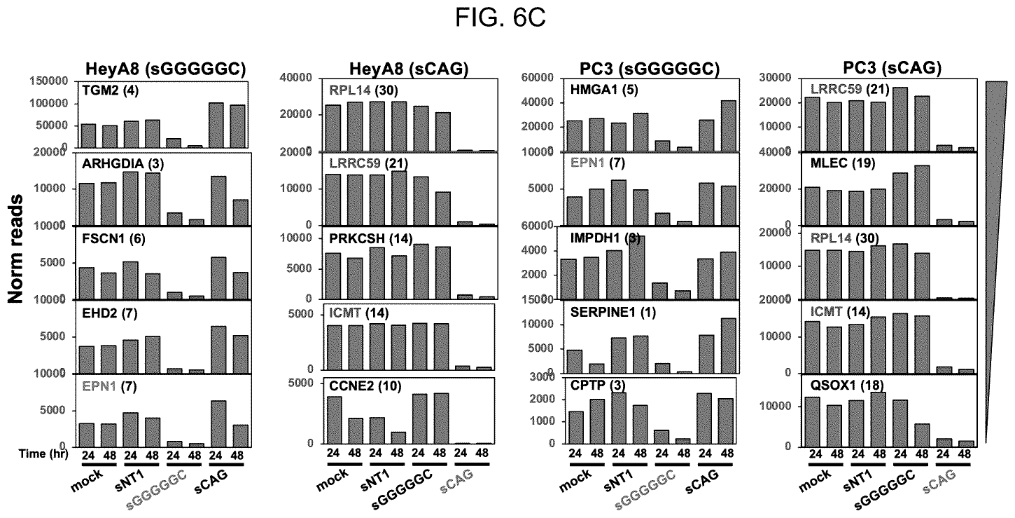

To identify genes that were significantly deregulated in the transfected cells the two samples harvested at each time point were treated as duplicates and expression differences between sNT1 versus sGGGGGC and sNT1 versus sCAG were determined for both cell lines using EdgeR. These four lists ranked from highest to lowest fold downregulation were the subjected to a gene set enrichment analysis (GSEA) using the gene lists of putative targets described above. As shown in FIG. 6A, in all cases we detected a substantial enrichment of the genes carrying the predicted target sites in the most downregulated genes suggesting that they are targeted by the transfected sRNA. It is also important to note that neither sGGGGGC nor sCAG has any toxic activity to cells that are deficient in Ago2 ((6) and data not shown). Hence, the toxic activity is completely dependent on RNAi. We recently confirmed that sGGGGGC does indeed act like a miRNA by targeting the seed GCCCCC predominantly when located in the 3′UTR of targets (2). To assemble a list of genes for each cancer cell line that are targeted of either of the two sRNAs, we identified the target containing genes that are significantly expressed and ranked them according to highest downregulation upon sRNA transfection. We excluded all genes that were 1.5, or more, -fold downregulated by the other toxic sRNA to only identify genes that were selectively targeted by either one of the two toxic sRNAs. The top ten genes were selected and again ranked according to highest expression (FIG. 6B).

Of these genes we are showing the normalized reads of the top five genes for each transfected cell line and sRNA (FIG. 6C). Each of these genes was fundamentally and selectively downregulated in the cells treated with the toxic sRNA and all 20 were slightly more profoundly downregulated at 48 hrs compared to 24 hrs. This suggests ongoing and sustained RNAi. We also noticed that the extent of target silencing was more pronounced in the cells treated with sCAG compared to the ones transfected with sGGGGGC, consistent with the much higher potency of sCAG. Note, that of the five highly abundant CUG target containing and most downregulated (by sCAG) genes 3 were identified in both cell lines (labeled in green in FIG. 6B) and one of them, RLP14, is part of our curated list of critical survival genes (8). These last analyses are important to analyze tumors in treated mice. They will allow us to use simple qPCR analysis to confirm target engagement.

### 2.4. Evidence of Target Engagement in Ovarian and Prostate Cancer

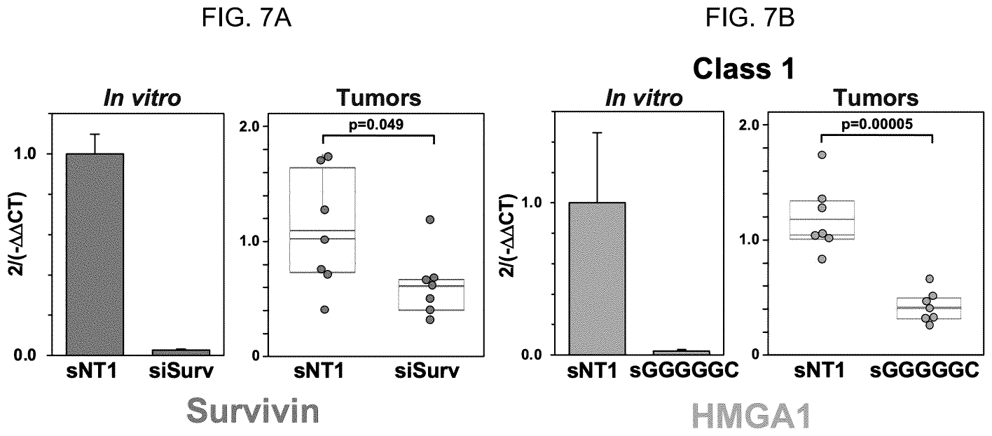

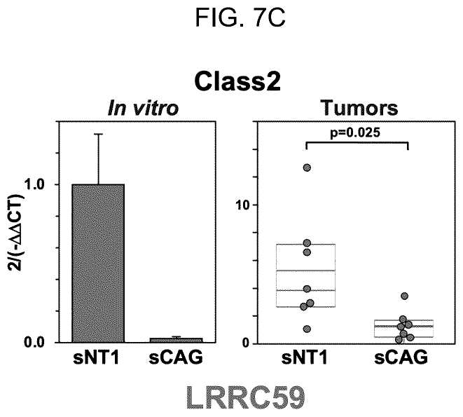

Now that we had identified targets for both sGGGGGC and sCAG in cancer cells, we could determine whether the LPP-sRNAs reached their targets in the prostate cancer tumors in the treated mice. We chose to analyze expression of Survivin, the most abundant target of sGGGGGC, HMGA1 (see FIG. 6C) and the most abundant target for sCAG, LRRC59. All sRNAs were highly active as evidenced by the efficient knockdown of their targeted genes in in vitro transfected PC3 cells. All three targets were significantly downregulated in the tumors treated with the targeting sRNA when compared to the nontargeting sNTI delivered using LPPs (FIG. 7). It is remarkable that we detected such a profound knockdown of targeted genes in tumors that had been treated 6 times with the LPP-sRNAs over the course of two weeks. That suggests the LPP particles efficiently deliver the sRNAs in a therapeutic setting, continue to have access to the tumor cells and that RNAi remains active and the targets susceptible to RNAi in the treated tumors.

### 2.5 No Changes to the Bone Marrow in Immune Competent Mice Bearing Orthotopically Implanted with Syngeneic OC Tumors Treated with LPP-sGGGGGC

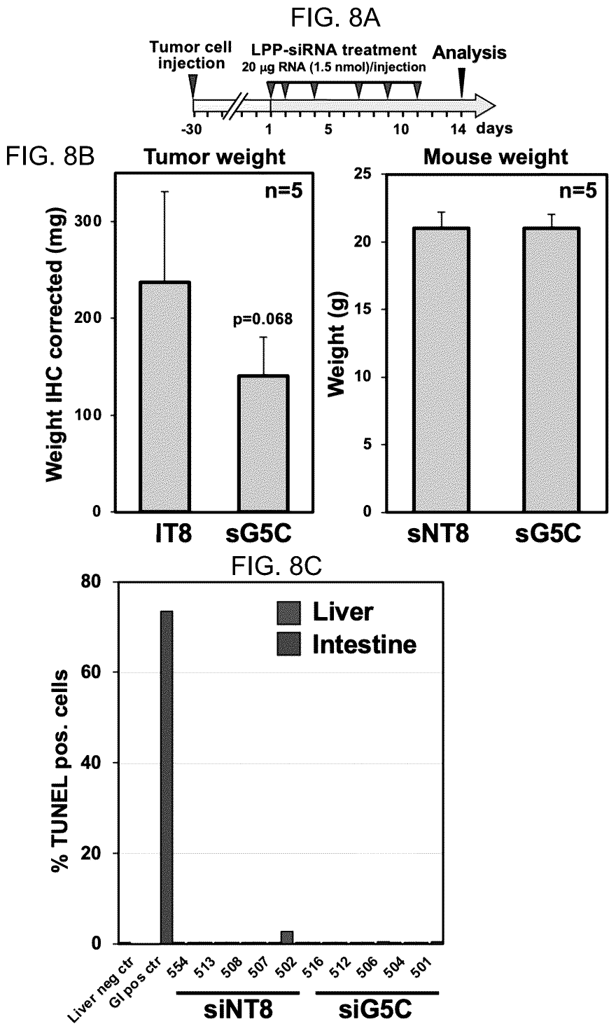

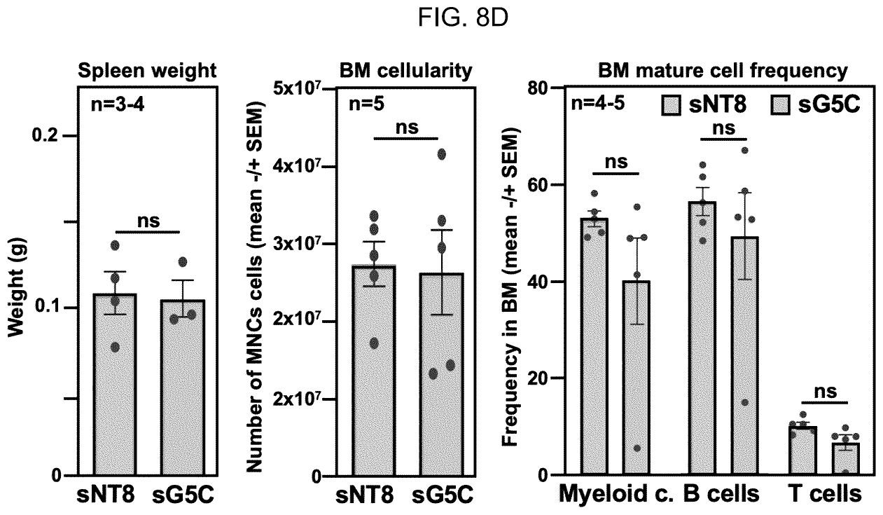

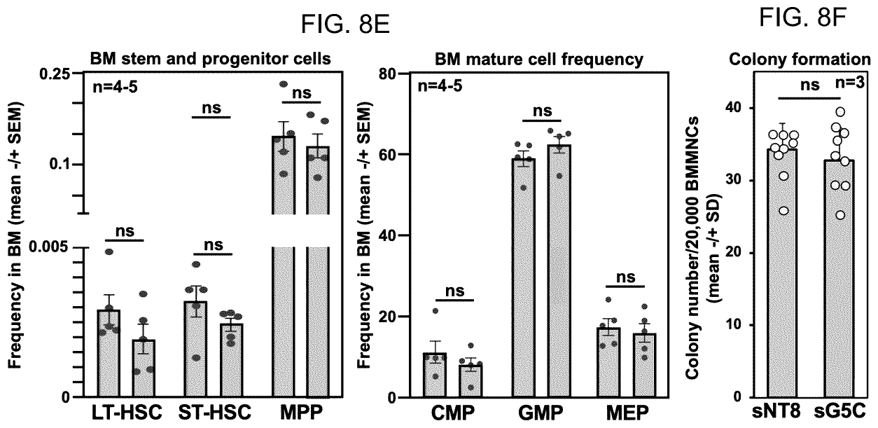

To test whether the treatment with LPP-sGGGGGC would also affect the growth of orthotopically grown tumor cells in immune competent mice, we injected C57BL/6 mice with the aggressive OC cell line ID8 rendered highly similar to human HGSOC by deleting both p53 and BRCA2 (27). After one month of tumor implantation treatment was given three times a week for two weeks (FIG. 8A). Two days after the last treatment tumors and mice were analyzed. Tumor weight in the LPP-sGGGGGC (sG5C) treated mice were reduced about ˜40% (FIG. 8B). While this is not a complete remission, we need to point out that this pilot experiment was first designed to test for toxicity of the particles on bone marrow (BM) cells. Important in this experiment, we again saw no signs of liver toxicity (FIG. 8C). The standard treatment for OC is chemotherapy which has profound side effects including in the hematopoietic system and the BM (28, 29). A comprehensive analysis of the hematopoietic system did not reveal a significant difference in the number of immune cells, hematopoietic stem or progenitor cells (FIG. 8D, E). Finally, we tested the ability of the BM cells from the treated mice to form colonies on methyl cellulose (FIG. 8F). We observed only minor differences that were not statistically significant and are expected to be transient.

### 2.6 No Toxicity in Immune Competent Rats Orthotopically Implanted with Liver Cancer Cells and Treated with LPP-sGGGGGC

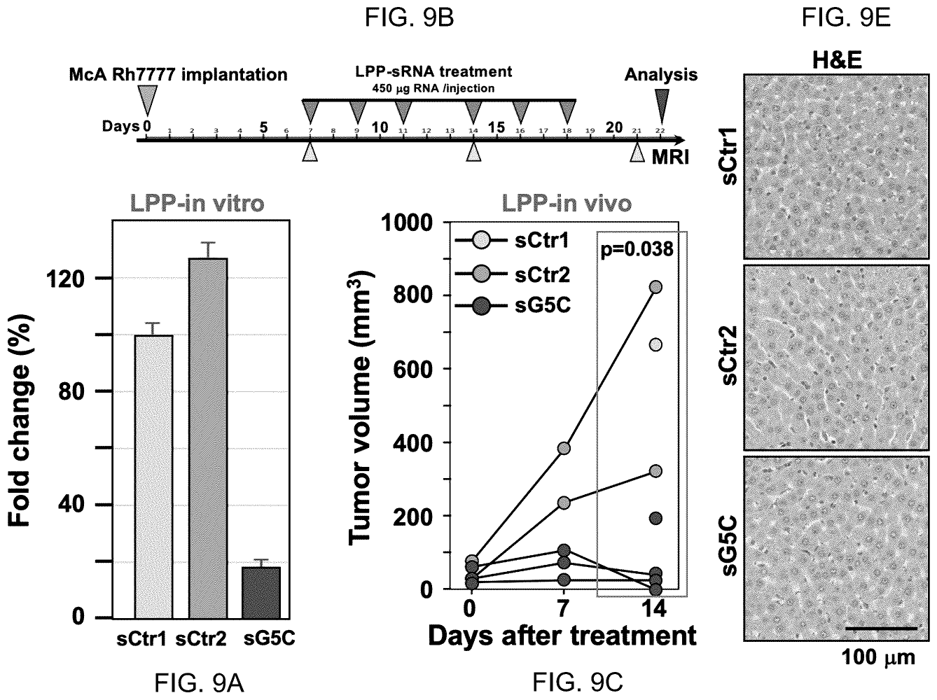

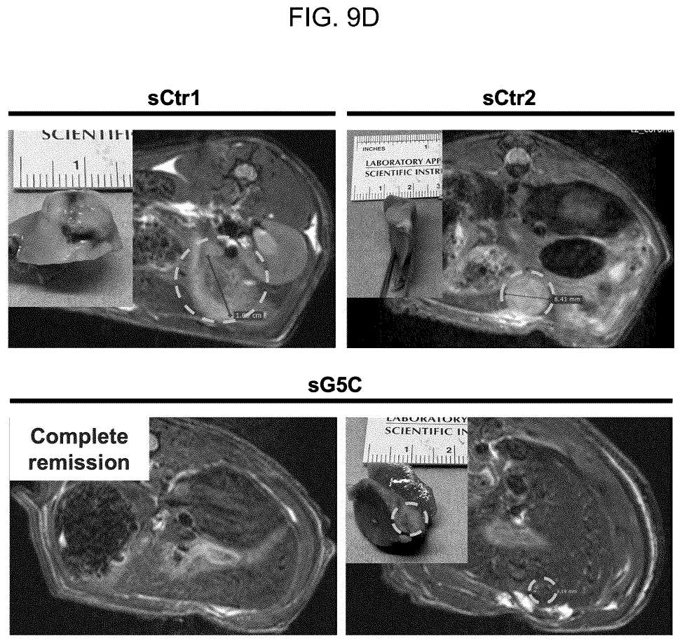

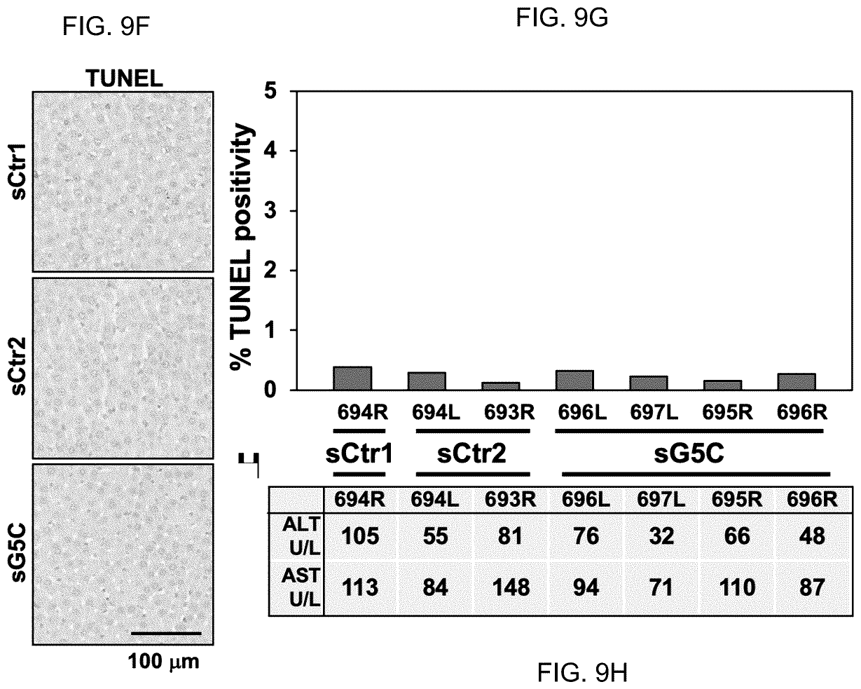

The toxicology of new therapeutic reagents is often done in rats. First, we tested the rat hepatoma cell line McA Rh7777 (McA) cells) which can be grown in Sprague Dawley rats (30) with LPP-siGGGGGC in vitro to establish that the sRNA could also kill rat cells. As expected McA cells were highly susceptible to the treatment with LPP-siGGGGGC when compared to two control sRNAs that were not toxic to this cell line (FIG. 9A). 2.5 million McA cells were then surgically implanted into the rat livers as described before (30-32), and a week after implantation, the size of tumors in the liver was measured by MRI. After 6 treatments with the LPPs-sRNAs (and 3 rounds of MRI) (FIG. 9B) rats were analyzed. Strikingly, the results were very similar to the in vitro treatment. While the control treated tumors kept growing, tumors in all rats treated with LPP-sG5C had their tumor sizes substantially reduced with one complete remission (FIG. 9C, D). The results were so fundamental that even though a small number of rats was used in this study the difference between the treatments reached statistical significance at end point when the 3 control and 4 LPP-sG5C treated tumors were compared (p=0.038). Again, no signs of toxicity were seen, body weight of the rats was not affected (not shown), liver histology was normal (FIG. 9E), TUNEL positivity in the liver was not elevated in the LPP-sG5C treated rats (FIG. 9F, G), and liver enzymes were normal (FIG. 9H).

### 2.7 Materials and Methods

### Synthesis of Tyrosine-Modified Branched PEI

In an example, 10 kDa branched PEI (0.35 g, 8.13 mmol in monomer, Polysciences Inc., Warrington, USA) was dissolved in 3 mL dry dimethylformamide in a glass vial under a nitrogen atmosphere with diisopropylamine (0.5 eq compared to PEI monomer: 0.69 mL, 4.06 mmol, Carl Roth, Karlsruhe, Germany). In a separate vial, N-Boc protected L-tyrosine (0.4 eq compared to PEI monomer: 0.91 g, 3.25 mmol, Carbolution Chemicals, St. Ingbert, Germany), N-hydroxysuccinimide (0.5 eq compared to PEI monomer: 0.47 g, 4.06 mmol, Carbolution Chemicals, St. Ingbert, Germany) were dissolved in 5 mL dry dimethylformamide in a glass vial under a nitrogen atmosphere. Diisopropylamine (0.5 eq compared to PEI monomer: 0.69 mL, 4.06 mmol, Carl Roth, Karlsruhe, Germany) was added. Under stirring, 1-Ethyl-3-(3-dimethylaminopropyl) carbodiimide (EDC x Cl) (0.5 eq compared to PEI monomer: 0.78 g, 4.06 mmol, Carbolution Chemicals, St. Ingbert, Germany) was added in portions and stirred for 3 h. The pre-activated tyrosine mixture was added to the PEI solution and further stirred for 3 days at room temperature under a nitrogen atmosphere. The crude reaction mixture was purified by dialysis (3.5 kDa MWCO, regenerated cellulose, Serva, Heidelberg, Germany) against methanol for 6 h with intermediate solvent replacement to remove by-products and dimethylformamide. The methanol was removed in vacuo and the polymer was dissolved in 5 mL trifluoroacetic acid and stirred overnight for Boc-deprotection. Excess trifluoroacetic acid was removed by co-evaporation with ethanol. Finally, the tyrosine-modified 10 kDa PEI (P10Y) was dissolved in distilled water and purified by dialysis against 0.05 M HCl for 24 h, then by dialysis against distilled water for 48 h with intermediate solvent replacement. Lyophilization yielded P10Y as white to yellowish fluffy powder. The degree of functionalization was confirmed by 1H-NMR and is in the range of 30-35% compared to the total monomer content.

### Synthesis of Tyrosine-Modified Linear PEI

In an example, 10 kDa linear PEI (0.2 g, 4.64 mmol in monomer, Sigma Aldrich, Taufkirchen, Germany) was dissolved in 5 mL dry dimethylformamide in a glass vial under a nitrogen atmosphere with diisopropylamine (1.5 eq compared to PEI monomer: 1.22 mL, 6.96 mmol, Carl Roth, Karlsruhe, Germany) and heated to 65° C. to completely dissolve the polymer. In a separate vial, N-Boc protected L-tyrosine (0.4 eq compared to PEI monomer: 0.522 g, 1.86 mmol, Carbolution Chemicals, St. Ingbert, Germany) was dissolved in 5 mL dry dimethylformamide in a glass vial under a nitrogen atmosphere. PyBOP (0.5 eq compared to PEI monomer: 1.21 g, 2.32 mmol, Carbolution Chemicals, St. Ingbert, Germany) was added in portions and stirred for 30 min. The pre-activated tyrosine mixture was added to the pre-heated PEI solution dropwise. Next, the reaction mixture was stirred at room temperature. After 12 h, a second portion of PyBOP (0.1 eq compared to PEI monomer:0.24 g, 0.46 mmol) was added and further stirred for 2.5 days at room temperature under a nitrogen atmosphere. The crude reaction mixture was purified by dialysis (3.5 kDa MWCO, regenerated cellulose, Serva, Heidelberg, Germany) against methanol for 6 h with intermediate solvent replacement to remove by-products and dimethylformamide. The methanol was removed in vacuo and the polymer was dissolved in 5 mL trifluoroacetic acid and stirred overnight for Boc-deprotection. Excess trifluoroacetic acid was removed by co-evaporation with ethanol. Finally, the tyrosine-modified 10 kDa linear PEI (LP10Y) was dissolved in distilled water and purified by dialysis against 0.05 M HCl for 24 h, then by dialysis against distilled water for 48 h with intermediate solvent replacement. Lyophilization yielded LP10Y as white to yellowish fluffy powder. The degree of functionalization was confirmed by 1H-NMR and is in the range of 30-35% compared to the total monomer content.

### Preparation of sRNA Polyplexes

The polyplexes were prepared based on polymer/sRNA mass ratios. Typically, a mass ratio of 7.5 was used with the unmodified 10 kDa branched PEI and in the case of the tyrosine-modified PEIs (P10Y and LP10Y), a mass ratio of 2.5 was used.

For one single injection in mice, an appropriate amount of sRNA, e.g., 20 μg (1500 pmol), was diluted in 75 μL buffer. Prior to complexation, 10 kDa PEI (‘P10’) was purified by dialysis against distilled water. For complexation with the polymers P10 and P10Y, HBG buffer (5% glucose (w/v), 10 mM HEPES pH 7.4) was used, and for LP10Y, HBT buffer (10% trehalose (w/v), 20 mM HEPES pH 7.4) was employed. Separately, the optimal amount of polymer was diluted in 75 μL of the same buffer. Based on the optimal mass ratios, 150 μg of the unmodified 10 kDa branched PEI (P10) or 50 μg of P10Y/LP10Y were used. Next, the sRNA dilution was added to the polymer dilution and thoroughly mixed and incubated for 30 min at room temperature. The final volume for a single injection was 150 μL.

### Preparation of Lipopolyplexes

First, the polyplexes were prepared essentially as described above. In an example, 20 μg (1500 pmol) sRNA were diluted in 60 μL of buffer. In a separate vial, the calculated amount of polymer as detailed above was diluted in 60 μL buffer. The sRNA dilution was added to the polymer dilution, mixed thoroughly and incubated for 30 min at room temperature. For preparing the lipopolyplexes, optimal DPPC liposome amounts were diluted in 30 μL buffer. The calculation based on liposome/polymer mass ratios and was 5 for P10-based polyplexes and a mass ratio of 10 for P10Y- and LP10Y-based polyplexes.

30 μL of the DPPC liposomes were added to the polyplexes and mixed well by rapid pipetting. The mixture was then transferred to an ultrasound bath and sonicated for 5 min and at 50° C. Finally, the lipopolyplexes were incubated for at least 1 h at room temperature prior to use. The final volume for a single injection into mice was 150 μL.

### Preparation of the Liposomes

The liposomes were prepared using the “Thin-Film-Hydration-Extrusion” method. First, dipalmitoylphosphatidylcholine (DPPC, Avanti Polar Lipids, Alabaster, USA) 0.5 g were dissolved in chloroform/methanol (2:1, v/v) in a 50 mL round bottom flask. The solvent was evaporated on a rotary evaporator using a programmable vacuum pump and well-defined time/pressure steps (0 s/1000 mbar, 30 s/800 mbar, 5 min/500 mbar, 30 min/0 mbar). The flask with the lipid film was additionally dried overnight under high vacuum to ensure complete removal of solvents. The lipid film was hydrated using 20 mL HBT buffer (10% trehalose, 20 mM HEPES pH 7.4) and sonicated in an ultrasound bath at 50° C. until a homogeneous suspension was obtained. Thereafter, the suspension was extruded eleven times through a 200 nm polycarbonate membrane using a lipid extruder (Avanti Polar Lipids, Alabaster, USA).

### REFERENCES

1. Wach S, Brandl M, Borchardt H, Weigelt K, Lukat S, Nolte E, Al-Janabi O, Hart M, Grasser F, Giedl J, Jung R, Stohr R, Hartmann A, Lieb V, Hobel S, Peters A, Staubert C, Wullich B, Taubert H, Aigner A. (2019). Exploring the MIR143-UPAR Axis for the Inhibition of Human Prostate Cancer Cells In vitro and In vivo. Mol Ther Nucleic Acids. 16:272-83.
2. Patel M, Bartom E T, Paudel B, Kocherginsky M, O''Shea K L, Murmann A E, Peter M E. (2020). Identification of the toxic 6mer seed consensus in human cancer cells. BioRxiv.https://doi.org/10.1101/2020.12.22.424040.
3. Putzbach W, Gao Q Q, Patel M, Haluck-Kangas A, Murmann A E, Peter M E. (2018). DISE-A Seed Dependent RNAi Off-Target Effect that Kills Cancer Cells. Trends in Cancer. 4:10-9.
4. Patel M, Peter M E. (2018). Identification of DISE-inducing shRNAs by monitoring cellular responses. Cell Cycle. 17: 506-14.
5. Murmann A E, Yu J, Opal P, Peter M E. (2018). Trinucleotide repeat expansion diseases, RNAi and cancer. Trends in Cancer. 4:684-700.
6. Murmann A E, Gao Q Q, Putzbach W T, Patel M, Bartom E T, Law C Y, Bridgeman B, Chen S, McMahon K M, Thaxton C S, Peter M E. (2018). Small interfering RNAs based on huntingtin trinucleotide repeats are highly toxic to cancer cells. EMBO Rep. 19:e45336.
7. Gao Q Q, Putzbach W, Murmann A E, Chen S, Ambrosini G, Peter J M, Bartom E, Peter M E. (2018). 6mer seed toxicity in tumor suppressive miRNAs. Nature Comm. 9:4504.
8. Putzbach W, Gao Q Q, Patel M, van Dongen S, Haluck-Kangas A, Sarshad A A, Bartom E, Kim K Y, Scholtens D M, Hafner M, Zhao J C, Murmann A E, Peter M E. (2017). Many si/shRNAs can kill cancer cells by targeting multiple survival genes through an off-target mechanism. eLife. 6: e29702.
9. Murmann A E, McMahon K M, Halluck-Kangas A, Ravindran N, Patel M, Law C, Brockway S, Wei J J, Thaxton C S, Peter M E. (2017). Induction of DISE in ovarian cancer cells in vivo. Oncotarget. 8:84643-58.
10. Haluck-Kangas A, Patel M, Paudel B, Vaidyanathan A, Murmann A E, Peter M P. (2021). DISE/6mer Seed Toxicity-A powerful anti-cancer mechanism with implications for other diseases. J Exp Clin Cancer Res. In press.
11. Hadji A, Ceppi P, Murmann A E, Brockway S, Pattanayak A, Bhinder B, Hau A, De Chant S, Parimi V, Kolesza P, Richards J S, Chandel N, Djaballah H, Peter M E. (2014). Death induced by CD95 or CD95 ligand elimination. Cell Reports. 10:208-22.
12. Murmann A E, Bartom E T, Schipma M J, Vilker J, Chen S, Peter M E. (2019). 6mer seed toxicity in viral microRNAs. iScience. 23: 100737.
13. Aigner A. (2007). Applications of RNA interference: current state and prospects for siRNA-based strategies in vivo. Appl Microbiol Biotechnol. 76:9-21.
14. Hobel S, Koburger I, John M, Czubayko F, Hadwiger P, Vornlocher H P, Aigner A. (2010). Polyethylenimine/small interfering RNA-mediated knockdown of vascular endothelial growth factor in vivo exerts anti-tumor effects synergistically with Bevacizumab. J Gene Med. 12:287-300.
15. Weirauch U, Beckmann N, Thomas M, Grunweller A, Huber K, Bracher F, Hartmann R K, Aigner A. (2013). Functional role and therapeutic potential of the pim-1 kinase in colon carcinoma. Neoplasia. 15:783-94.
16. Werth S, Urban-Klein B, Dai L, Hobel S, Grzelinski M, Bakowsky U, Czubayko F, Aigner A. (2006). A low molecular weight fraction of polyethylenimine (PEI) displays increased transfection efficiency of DNA and siRNA in fresh or lyophilized complexes. J Control Release. 112:257-70.
17. Ibrahim A F, Weirauch U, Thomas M, Grunweller A, Hartmann R K, Aigner A. (2011). MicroRNA Replacement Therapy for miR-145 and miR-33a Is Efficacious in a Model of Colon Carcinoma. Cancer Res. 71:5214-24.
18. Borchardt H, Ewe A, Morawski M, Weirauch U, Aigner A. (2021). miR24-3p activity after delivery into pancreatic carcinoma cell lines exerts profound tumor-inhibitory effects through distinct pathways of apoptosis and autophagy induction. Cancer Lett. 503:174-84.
19. Linder B, Weirauch U, Ewe A, Uhmann A, Seifert V, Mittelbronn M, Harter P N, Aigner A, Kogel D. (2019). Therapeutic Targeting of Stat3 Using Lipopolyplex Nanoparticle-Formulated siRNA in a Syngeneic Orthotopic Mouse Glioma Model. Cancers (Basel). 11.
20. Ewe A, Panchal O, Pinnapireddy S R, Bakowsky U, Przybylski S, Temme A, Aigner A. (2017). Liposome-polyethylenimine complexes (DPPC-PEI lipopolyplexes) for therapeutic siRNA delivery in vivo. Nanomedicine. 13:209-18.
21. Ewe A, Hobel S, Heine C, Merz L, Kallendrusch S, Bechmann I, Merz F, Franke H, Aigner A. (2017). Optimized polyethylenimine (PEI)-based nanoparticles for siRNA delivery, analyzed in vitro and in an ex vivo tumor tissue slice culture model. Drug Deliv Transl Res. 7:206-16.
22. Ewe A, Noske S, Karimov M, Aigner A. (2019). Polymeric Nanoparticles Based on Tyrosine-Modified, Low Molecular Weight Polyethylenimines for siRNA Delivery. Pharmaceutics. 11.
23. Ewe A, Przybylski S, Burkhardt J, Janke A, Appelhans D, Aigner A. (2016). A novel tyrosine-modified low molecular weight polyethylenimine (P10Y) for efficient siRNA delivery in vitro and in vivo. J Control Release. 230:13-25.
24. Karimov M, Schulz M, Kahl T, Noske S, Kubczak M, Gockel I, Thieme R, Buch T, Reinert A, Ionov M, Bryszewska M, Franke H, Krugel U, Ewe A, Aigner A. (2021). Tyrosine-modified linear PEIs for highly efficacious and biocompatible siRNA delivery in vitro and in vivo. Nanomedicine. 36:102403.
25. Hobel S, Aigner A. (2010). Polyethylenimine (PEI)/siRNA-mediated gene knockdown in vitro and in vivo. Methods Mol Biol. 623:283-97.
26. Corbin J M, Geordescu C, wren J D, Xu C, Asch A S, Ruiz-Echevarria M J. (2021). Seed-mediated RNA interference of androgen signaling and survival networks induces cell death in prostate cancer cells. Molecular Therapy Nucleic Acids. 24:337-51.
27. Walton J, Blagih J, Ennis D, Leung E, Dowson S, Farquharson M, Tookman L A, Orange C, Athineos D, Mason S, Stevenson D, Blyth K, Strathdee D, Balkwill F R, Vousden K, Lockley M, McNeish I A. (2016). CRISPR/Cas9-Mediated Trp53 and Brca2 Knockout to Generate Improved Murine Models of Ovarian High-Grade Serous Carcinoma. Cancer Res. 76:6118-29.
28. Grenman S E, Rantanen V T, Salmi T A. (1996). High-dose chemotherapy with autologous stem cell support in advanced ovarian cancer. Ann Med. 28:151-8.
29. ten Bokkel Huinink W W. (1991). Chemotherapy and complications in gynecologic cancer. Curr Opin Oncol. 3:930-2.
30. Kwak K, Yu B, Mouli S K, Larson A C, Kim D H. (2020). Sodium Cholate Bile Acid-Stabilized Ferumoxytol-Doxorubicin-Lipiodol Emulsion for Transcatheter Arterial Chemoembolization of Hepatocellular Carcinoma. J Vasc Interv Radiol. 31:1697-705 e3.
31. Choi B, Choi H, Kim H, Choi A, Kwon S W, Mouli S K, Lewandowski R J, Kim D H. (2022). Z-domain protein nano-bio interfaced MRI visible anti-program death ligand-1 nanoconjugates for enhanced local immune checkpoint inhibitor immunotherapy. Nano Today. 45:101552.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A composition comprising:
(1) a dsRNA having a first strand, otherwise referred to as an “A” strand, and a second strand, otherwise referred to as a “B” strand,
5′-A01 A02 A03 A04 A05 A06 A07 A08 A09 A10 A11 A12 A13 A14 A15 A16 A17 A18 A19 A20 A21-3′

| | | | | | | | | | | | | | | | | | | | |

3′-B21 B20 B19 B18 B17 B16 B15 B14 B13 B12 B11 B10 B09 B08 B07 B06 B05 B04 B03 B02 B01-5′
wherein the region A02-A07 of the dsRNA is GGGGGC and wherein the A and B strands comprise 3′ overhangs;
(2) a polyamine-based polymer, wherein the dsRNA is in complex with the polyamine-based polymer and forms particles.

2. The composition of claim 1, wherein the A and/or B strand comprises modified nucleotides.

3. The composition of claim 2, wherein the nucleotides at positions B01 and B02 are modified.

4. The composition of claim 3, wherein the modified nucleotides comprise 2′-O-methyl modifications.

5. The composition of claim 1, wherein the dsRNA and the polyamine-based polymer are present in polyplex particles.

6. The composition of claim 1, wherein the polyamine-based polymer comprises polyethyleneimine (PEI), optionally wherein the PEI is linear or branched, and wherein the average molecular weight of the polymer is 2,000-25,000 Da, preferably 5,000-20,000 Da.

7. The composition of claim 6, wherein the polyethyleneimine is modified with tyrosine.

8. The composition of claim 7, wherein 20-60%, 30-50%, or 30-35% of the primary amines in the polyethyleneimine are modified with tyrosine.

9. The composition of claim 1, wherein the composition further comprises (3) a lipid component, wherein the dsRNA and the polyamine-based polymer are present in lipopolyplexes (LPP).

10. The composition of claim 9, wherein the polyamine-based polymer comprises polyethyleneimine (PEI), wherein the PEI is linear PEI, wherein the average molecular weight of the PEI is 10 kDa, wherein the PEI is tyrosine modified, and wherein the lipid component comprises dipalmitoylphosphatidylcholine (DPPC).

11. The composition of claim 9, wherein the polyamine-based polymer comprises polyethyleneimine (PEI), wherein the PEI is branched PEI, wherein the average molecular weight of the PEI is 10 kDa, and wherein the lipid component comprises dipalmitoylphosphatidylcholine (DPPC).

12. The composition of claim 9, wherein the polyamine-based polymer comprises polyethyleneimine (PEI), wherein the PEI is branched PEI, wherein the average molecular weight of the PEI is 10 kDa, wherein the PEI is tyrosine modified, and wherein the lipid component comprises dipalmitoylphosphatidylcholine (DPPC).

13-18. (canceled)

19. A pharmaceutical composition comprising the composition of claim 9; and a pharmaceutically acceptable carrier, diluent, or excipient.

20. A method of treating a cell proliferative disease or disorder in a subject in need thereof comprising administering an effective amount of the pharmaceutical composition of claim 19 to treat the cell proliferative disease or disorder.

21. The method of claim 20, wherein the cell proliferative disease or disorder is ovarian cancer or prostate cancer.

22-23. (canceled)

24. A method of killing a cancer cell comprising contacting the composition of claim 1 to the cancer cell.

25. The method of claim 24, wherein the cancer cell is an ovarian cancer cell, or derived from an ovarian cancer cell, or wherein the cancer cell is a prostate cancer cell, or derived from a prostate cancer cell.

26. (canceled)

27. A composition comprising:
(1) a double-stranded polynucleotide comprising a passenger strand and a guide strand, the double-stranded polynucleotide comprising: a trinucleotide repeat sequence (X₁X₂X₃)ₙ, wherein the trinucleotide repeat of the guide strand is (CAG)ₙ, and wherein the guide and passenger strand comprise 3′ overhangs, and
(2) a polyamine-based polymer, wherein the dsRNA is in complex with the polyamine-based polymer and forms particles.

28-45. (canceled)

46. A method of treating a cell proliferative disease or disorder in a subject in need thereof comprising administering an effective amount of the composition of claim 27 to treat the cell proliferative disease or disorder.

47-49. (canceled)

50. A method of killing a cancer cell comprising contacting the composition of claim 27 to the cancer cell.

51-52. (canceled)


SEQUENCE LISTING (7 sequences)

SEQ ID NO: 1 - RNA, 24 nt, synthetic construct
  source 1..24: mol_type = other RNA
ctgctgctgctgctgctgctgctg

SEQ ID NO: 2 - RNA, 19 nt, synthetic construct
  source 1..19: mol_type = other RNA
  modified_base 19: mod_base = OTHER; note = deoxyadenosine
tggtttacatgtcgactaa

SEQ ID NO: 3 - RNA, 21 nt, synthetic construct
  source 1..21: mol_type = other RNA
ttagtcgacatgtaaaccaaa

SEQ ID NO: 4 - RNA, 19 nt, synthetic construct
  source 1..19: mol_type = other RNA
  modified_base 19: mod_base = OTHER; note = deoxyadenosine
tggtttacatgtgccccca

SEQ ID NO: 5 - RNA, 21 nt, synthetic construct
  source 1..21: mol_type = other RNA
tgggggcacatgtaaaccaaa

SEQ ID NO: 6 - RNA, 19 nt, synthetic construct
  source 1..19: mol_type = other RNA
  modified_base 19: mod_base = OTHER; note = deoxyguanosine
gctgctgctgctgctgctg

SEQ ID NO: 7 - RNA, 21 nt, synthetic construct
  source 1..21: mol_type = other RNA
cagcagcagcagcagcagcag